(19) United States
(12) Patent Application Publication
(10) Pub. No.: US 2022/0277813 A1
(43) Pub. Date: Sep. 1, 2022

(54) Effects of a Molecule

(71) Applicants: Kirill Veselkov, London (GB); Jozef Youssef, Barnet (GB); Ivan Loponogov, London (GB); Michael Bronstein, London (GB)

(72) Inventors: Kirill Veselkov, London (GB); Jozef Youssef, Barnet (GB); Ivan Loponogov, London (GB); Michael Bronstein, London (GB)

(21) Appl. No.: 17/622,179

(22) PCT Filed: Jul. 2, 2020
(86) PCT No.: PCT/GB2020/051591

§ 371 Date: Dec. 22, 2021

Related U.S. Application Data
(60) Provisional application No. 62/869,626, filed on Jul. 2, 2019.

(51) Int. Cl. G16B 40/30; G16B 5/20

(52) U.S. Cl. CPC G16B 40/30; G16B 5/20

(57) ABSTRACT

A method of identifying latent network-wide effects of a given molecule is disclosed. The method comprises receiving interaction data relating to interactions between a molecule(s) and/or a biomolecule(s) and/or a biological cell(s) and/or a biological process(es). The method further comprises generating an interactome network by mapping the molecule(s) and/or biomolecule(s) and/or biological cell(s) and/or biological process(es) interacting with input molecules onto a graph comprising node(s) and node link(s), wherein each node is a molecule(s) and/or a biomolecule(s) and/or a biological cell(s) and/or a biological process(es) and each node link corresponds to interactivity. The method further comprises generating a list of a molecule(s) and/or a biomolecule(s) and/or a biological cell(s) and/or a biological process(es) found in the interactome network that are affected by a given input molecule by using unsupervised learning on graphs to identify latent network-wide effects of the given input molecule.

### FIELD

The present invention relates to identifying network-wide effects of a molecule.

### BACKGROUND

With rapidly ageing populations, the world is experiencing an unsustainable healthcare and economic burden from chronic diseases such as cancer, cardiovascular, metabolic and neurodegenerative disorders. Diet and nutritional factors play an essential role in the prevention of these diseases and significantly influence disease outcome in patients during and after therapy. According to most recent data, up to 30-40% of all cancers can be prevented by dietary and lifestyle modifications alone. Plant-based foods (i.e. derived from fruits and vegetables) are particularly rich in cancer-beating molecules (CBM) such as polyphenols, flavonoids, terpenoids and botanical polysaccharides. Evidence from experimental studies has implicated multiple mechanisms of action by which dietary agents contribute to the prevention or treatment of various cancers. These include regulating the activity of inflammatory mediators and growth factors, suppressing cancer cell survival, proliferation, and invasion, as well as angiogenesis and metastasis.

Being able to first identify food ingredients and later design “hyperfoods” that are richest in CBMs and having health promoting or therapeutic influence, represents an unprecedented opportunity to reduce healthcare costs and potentially enhance health outcomes for chronic diseases such as cancer. Since in the modern era of designer gastronomy the consumers are increasingly discerning and demanding, the design of hyperfoods is a multi-faceted optimization problem taking into account not only pro-health benefits, but also considering various aesthetic (e.g. color, texture) and sensory (e.g. taste, mouthfeel) characteristics. We argue that at least some parts of such design could be performed computationally, by exploiting artificial intelligence (AI) technology. As outlined in our recently published 10-point manifesto (‘The Future of Computing and Food’), this will require a collaborative approach of multiple stakeholders including food producers, chefs, designers, engineers, data scientists, sensory scientists and clinicians.

### SUMMARY

According to a first aspect of the invention there is provided a computer-implemented method. The method comprises receiving interaction data relating to interactions between a molecule(s) and/or a biomolecule(s) and/or a biological cell(s) and/or a biological process(es). The method further comprises generating an interactome network by mapping the molecule(s) and/or biomolecule(s) and/or biological cell(s) and/or biological process(es) interacting with input molecule(s) onto a graph comprising node(s) and node link(s), wherein each node is a molecule (s) and/or a biomolecule(s) and/or a biological cell(s) and/or a biological process(es) and each node link corresponds to interactivity. The method further comprises generating a list of a molecule(s) and/or a biomolecule(s) and/or a biological cell(s) and/or a biological process(es) found in the interactome network that are affected by a given input molecule by using unsupervised learning on graphs to identify latent network-wide effects of the given input molecule.

The molecule(s) or input molecule(s) may be organic or inorganic. The molecule(s) or input molecules may be or be a component(s) of a (known or unknown) drug(s) or biological organisms. The molecule(s) or input molecule(s) may be or be a component(s) of a (known or unknown) plant(s), fungus/fungi or food(s) or foodstuff(s) mineral(s). The molecule(s) or input molecule(s) may be or be a component(s) of a (known or unknown) functional food(s), dietary supplement(s) or nutraceutical(s).

The molecule(s) which may be used to generate the interactome may be the same molecule(s) as the input molecule(s). For example, the molecule glucose may be mapped onto an interactome together with other molecule(s) and/or biomolecule(s) and/or biological cell(s) and/or biological process(es). The latent network-wide effects of glucose may then be identified.

Many molecules within drugs exert their biomedical and functional activity by binding to a specific subset of biomolecules, e.g. proteins. Biomolecules, e.g. proteins rarely function in isolation but rather operate as part of highly interconnected networks. This method allows the use of unsupervised learning on graphs to simulate the down-stream influence of molecules on proteome networks (e.g. human, animal, plant or microbe proteome networks) from “sparse” protein target datasets. This network diffusion transforms a short list of proteins (the sparse protein target datasets) targeted by a given molecule or drug into a genome-wide profile of gene scores based on their network proximity to target candidates. Once the network has been generated, it is possible to simulate the perturbation of individual molecules on the proteome networks. This may provide information as to how the molecule, or combination of molecules interacts with a biological system or a component of a biological system (e.g. human organism or biomolecule pathway).

Interaction data may include interaction data between a molecule(s) and a molecule(s), interaction data between a molecule(s) and a biomolecule(s), interaction data between a molecule(s) and a biological cell(s), or a molecule(s) and interaction data between a biological process(es). Interaction data may include interaction data between a biomolecule(s) and a biomolecule(s), interaction data between a biomolecule(s) and a biological cell(s) or interaction data between a biomolecule(s) and a biological process(es). Interaction data may include interaction data between a biological cell(s) and a biological cell(s), interaction data between a biological cell(s) and a biological process(es). Interaction data may include interaction data between a biological process(es) and a biological process(es). Interaction data may further include interaction data between a biological entity/entities and a biological entity/entities, interaction data between a biological entity/entities and a molecule, interaction data between a biological entity/entities and a biomolecule(s), interaction data between a biological entity/entities and a biological cell(s) and interaction data between a biological entity/entities and a biological process(es). Interaction data may also include interactions between one or more element(s), for example, hydrogen, iron, zinc or lithium, and any one or combination of a molecule(s), biomolecule(s), a biological cell(s) or a biological process(es).

A biomolecule to biomolecule interaction may be, for example, a protein or enzyme acting on a carbohydrate, such as amylase acting on starch. An example of a molecule to biomolecule interaction may be a molecule in a pharmaceutical drug binding to a protein. An example of a biomolecule(s) interacting with a biological cell(s) may be vitamin D interacting with a dendritic cell and/or a macrophage, or thyroxin interacting with a cell membrane.

An example of a biological process(es) interacting with a molecule(s) or biomolecule(s) may be a vitamin modulating or disrupting a metabolism or other physiological process.

An example of a biological cell interacting with another biological cell may be biological cells forming cell-cell junctions.

Interaction data may be in vivo interaction data. Interaction data may be in vitro interaction data. Interaction data may be interaction data related to a biological process(es).

An interactome may comprise a molecule(s) and/or biomolecule(s) and/or biological cell(s) and/or a biological process(es) interaction graph.

A biomolecule may be, for example, a carbohydrate, a protein, a nucleic acid or a lipid. A biomolecule may be, for example, a gene, a protein or a metabolite. Biomolecules may include, for example, a group of genes, proteins or metabolites, or a mixture or combination of these. A biological process(es) may include, for example, a biomolecule pathway(s), a biomolecule super-pathway(s) or a gene ontology/ontologies. A biological cell(s) may be, for example a prokaryote(s) or a eukaryote(s). A biological cell(s) may be a microbe(s) in a microbiome(s). A collection of biological cells may form a tissue or tissues.

An interaction between a biomolecule(s) and/or process(es) involving a biomolecule(s) and/or a biological process(es) and a molecule(s) may include protein binding.

The latent network-wide effects of a given input molecule(s) may comprise biomolecule(s) binding affinity.

The interactome may include edge features representing the interactions between pairs of biomolecules and/or processes involving a biomolecule(s) and/or node features representing the biomolecule(s) and/or process(es) involving a biomolecule.

The interaction data relating to interactions between an input molecule(s) and a molecule(s) and/or a biomolecule(s) and/or a biological process(es) may include a molecule(s) interaction signal.

The method may further comprise generating an input molecule(s) interaction descriptor. Generating an input molecule(s) interaction descriptor may comprise applying a diffusion kernel to an input molecule(s) interaction data and/or signal on the biomolecule and/or the biomolecule pathway interaction graph and/or applying at least one layer of graph convolutional neural network (CNN) to the input molecule(s) interaction data and/or signal on the interactome.

The interactivity may be, for example, biological or chemical interactivity.

A biological process(es) may be a process(es) involving a biomolecule(s).

The type of interactome network may be experimentally derived and/or computationally predicted.

An example of an experimentally derived network is BioPlex. An example of a computationally predicted and experimentally derived network is STITCH.

The unsupervised learning on graphs may be a random walk with a diffusion kernel or operator.

The diffusion kernel or operator may be linear or non-linear. The diffusion kernel or operator may be restarts.

The unsupervised learning on graphs may further comprise varying a parameter(s) of the interactome and varying a parameter(s) of diffusion algorithms.

For example, the unsupervised learning on graphs may comprise varying a connection threshold(s) of the node link(s) and/or varying the probability of the random walk(s) restarting.

The method may further comprise generating a genome-wide profile of gene scores based on gene interactome network proximity to an input molecule(s) target candidates.

The entry node for a random walk represents a targeted molecule(s) and/or a targeted biomolecule(s) and/or a targeted biological cell(s) and/or a targeted biological process(es).

The targeted biomolecule may represent a targeted protein. The target biological cell may represent, for example, a cell in a microbiome. The biological process(es) may represent a, or part of a, metabolic or biochemical pathway.

The method may further comprise simulating the perturbation of one or more input molecule(s) through the interactome network using the input molecule(s) interaction data and outputting the interactions the of the input molecule(s) in the network.

The input molecule(s) may be a molecule(s) in an existing drug(s) or a bioactive compound(s) in food.

The method may further comprise generating a sparse molecules(s) and/or biomolecule(s) and/or biological cell(s) and/or biological process(es) profile interacting with an input molecule by assigning a value of 1 to all molecules(s) and/or biomolecule(s) and/or biological cell(s) and/or biological process(es) in the interactome that interact with the input molecule and assigning a value of 0 to all other molecules(s) and/or biomolecule(s) and/or biological cell(s) and/or biological process(es).

According to a second aspect of the invention, there is provided a computer implemented method. The method comprises receiving a list of a molecule(s) and/or a biomolecule(s) and/or a biological cell(s) and/or a biological process(es) found in an interactome network that are affected by a plurality of input molecules, each input molecule in a sub-set of the plurality of input molecules being identified as an anti-target input molecule or a non-anti-target input molecule. The method further comprises for a predetermined target, generating a trained model using supervised machine learning to classify input molecules as either anti-target or non-anti-target based on the influence of the input molecules on the interactome network.

The target may be a biological process(es), such as a biochemical process(es) or pathway or a process(es) involving a biomolecule or biomolecule pathway, or a chemical process or pathway. The target may be a phenotypic feature. The term “phenotypic feature” means an identifiable trait, condition or disease. It includes observable characteristics, such as one or more aspects of morphology, for example the size or shape of an appendage; physiology, for example ability to metabolise a particular chemical or the metabolic rate; or behaviour, such as aggression. It also includes diseases, clinical conditions and/or pathologies in any stage or state, or a marker of a disease, clinical condition or pathology, or a marker of a response to treatment of a disease. It also includes desirable traits (for example increased grain yield in wheat), or undesirable traits, such as biofilm formation in a bacteria or bacterial resistance to an antibiotic.

The phenotypic feature may be a disease, clinical condition or pathology, or a stage of a disease, clinical condition or pathology; or a marker of a disease, clinical condition or pathology. If the phenotypic feature is a disease, the disease may be, for example, cancer, diabetes, or depression. Alternatively, the phenotypic feature may be a marker of a response to treatment of a disease, clinical condition or pathology or a stage of a disease, clinical condition or pathology. Examples include elevation of one or more markers of inflammation; depression of a metabolite or hormone, for example depression of insulin levels as an indicator of diabetes; presence or absence of biomarkers associated with a disease or condition, for example CD34 or CD38 as prognostic biomarkers for acute B lymphoblastic leukemia; elevation or depression of expression of transcripts, proteins and/or metabolites, for example elevation of phospholipid metabolites as an indicator of cancer cell growth, or altered levels of cell death markers, such as apoptotic markers, as an indicator of neurodegenerative conditions or cancer.

The interactome network may comprise more than one interactome network. The interactome network may be a diffused interactome network.

The method may further comprise outputting molecule characteristics, such as how they interact with the interactome, which a biomolecule(s) and/or biological cell(s) and/or a biological process(es) they interact with and how they interact with them.

The influence of the input molecule(s) on an interactome network may be determined by applying at least one layer of parametric diffusion to the input molecule(s) data on the molecule(s) and/or biomolecule(s) and/or biological cell(s) and/or a biological process(es) interactome.

The parameters of parametric diffusion may be determined by training.

The training procedure may comprise receiving a training dataset of input molecule(s), the dataset, may comprise for each input molecule(s): a molecule interaction signal and the input molecule(s) ground-truth property for each molecule; tuning the parameters to optimize a loss function.

The training dataset of input molecule(s) may further include a molecule chemical descriptor for each input molecule(s).

The loss function may comprise at least one selected form the group of: a distance between the predicted input molecule(s) properties and the ground-truth input molecule(s) properties; or a classification error.

The training dataset may comprise a positive example(s) of an input molecule(s) or drugs efficient against a disease and negative examples of and input molecule(s) or drugs inefficient against a disease. The predicted input molecule(s) property may be efficiency against disease.

The supervised machine learning strategy may be based on Support Vector Machine model, SVM, Maximum Margin Criterion model, MMC, a convolutional neural network model, CNN, or a regularized LASSO/Elastic Net classifier algorithm.

If the strategy was based on an SVM model, the parameters for linear (“c”) and radial kernels (“c”, gamma) may be optimized during training.

The main measuring criterion for the performance of the model may be the F-score of the model's accuracy.

According to a third aspect of the invention, there is provided a computer implemented method. The method comprises receiving data identifying an input molecule(s) and/or characteristic(s) of the input molecule(s). The method further comprises receiving a trained supervised machine learning model, the trained model generated using a supervised machine learning strategy to classify an input molecule(s) as either anti-target or non-anti-target based on the influence of the input molecule(s) on an interactome network of a molecule(s) and/or a biomolecule(s) and/or a biological cell(s) and/or a biological process(es). The method further comprises, for a given target, determining, using the trained model, a prediction whether the input molecule(s) is an anti-target or a non-anti-target input molecule(s).

According to an aspect of the present invention there is provided a product formulated according to any one of or any combination of the methods. The product may comprise or include molecule(s) predicted by the method to have an anti-target effect, for example, an anti-disease effect. The product may include a dietary plan and/or supplement, for example a nutritional supplement or a food supplement, containing foods or foodstuffs which include molecule(s) predicted by the method to have an anti-target effect.

The method may further comprise outputting a product and/or dietary food plan formulated according to the method. The product and/or dietary food plan may be outputted to storage and/or it may be displayed and/or it may be transmitted.

The data identifying an input molecule(s) and/or characteristic(s) of the input molecule(s) may be structural data, bioinformatics data or data relating to how an input molecule(s) interacts with the interactome, proteome or genome. It may include the names of the proteins or genes an input molecule(s) interacts with, it may include the strength of the interaction between an input molecule(s) and proteins or genes.

With such information, it may be possible to use supervised machine learning, using the data of an input molecule with a confirmed specific target (e.g. an approved therapeutic drug), to identify different molecules which may have the same or similar targets. Thus, for example, known drugs with nationally approved status but approved for a different target, may be repurposed for a different use. Furthermore, molecules from other sources, for example flavour or colour molecules from foods and drink, may be identified as having the same of similar targets as a molecule with a known target. Using the genome-wide profiles of molecules within existing drugs, the supervised machine-learning model (e.g. “maximum margin criterion” or “support vector machines”) can be trained to accurately classify molecules with a specific target (for example those which may have anti-disease properties vs those without an identified specific target in the network and may have non-anti-disease properties). This supervised learning based on the on the influence of molecules on diffused interactome networks allows the identification of predictive (sub-)networks for anti-disease molecules.

The data identifying an input molecule(s) may include a molecule(s) interaction signal. An input molecule(s) interaction signal may comprise how an input molecule(s) interact(s) with one or more molecules(s) and/or biomolecules and/or one or more biological processes and/or one or more biological cell(s).

The data identifying an input molecule(s) may include a molecule(s) descriptor, which may be or include a chemical descriptor. The chemical descriptor may be obtained by applying a graph neural network to the interactome of the input molecule(s).

The influence of the input molecule(s) on an interactome network may be determined by applying at least one layer of parametric diffusion to the input molecule(s) data on the biomolecule interactome.

The prediction may include efficiency data against at least one target, for example, a disease type or cancer phenotype. The prediction may include toxicity data.

The parametric diffusion may be a random walk with a fixed transition matrix, diffusion process dependent on node and edge features, a graph attention diffusion or non-linear graph message passing.

Using the input molecule(s) data (e.g. molecule interaction descriptor) for determining, using the trained model, a prediction whether the input molecule(s) is an anti-target or a non-anti-target candidate molecule may comprise applying a neural network to the input molecule(s) data.

The influence of the input molecule(s) on an interactome network may further comprise pooling on the interactome. Pooling may comprise using a hierarchy of graphs obtained from the input interactome. The pooling may be learnable. Pooling may be applied to higher-level structures of molecule(s) and/or biomolecule(s) and/or biological cell(s), and/or biological process(es), for example biomolecule or biochemical pathways.

The data relating to the input molecule(s) may be interactome network-wide diffused effect data.

The data relating to an input molecule(s) may include a simulated perturbation of an input molecule(s) through interactome network-wide diffused effect data.

The method may further comprise calculating the anti-target probability outcome of the best performing learning strategy for a given input molecule(s).

The method may further comprise: for an input molecule determined as anti-target: extracting information relating to the input molecule(s) and information relating to the input molecule(s) therapeutic effects from a database using natural language processing; for the given target, determining whether the input molecule is a confirmed anti-target molecule. Determining whether the input molecule is a confirmed anti-target molecule may be performed by comparing information relating to the input molecule with the extracted information.

In this way, the best obtained models can then be used to predict the probability of a given existing approved drug to exhibit anti-disease properties. After validation of the predictive capacity of the model for anti-disease drug repositioning, the same machine learning strategy was applied to predict various cancer-beating molecules within foods.

The method may further comprise outputting a list of confirmed anti-target molecule(s).

Once an input molecules(s) is validated on anti-target (e.g. anti-disease or anti-cancer) therapeutics, compounds from other sources (for example, food and drink compounds) may be processed in exactly the same way as the molecules (e.g. therapeutic drugs and drug compounds) used to train the models. The best models may be used to generate probabilistic predictions for the anti-target “likeness” of these compounds.

The list of the compounds with the highest probability of exhibiting anti-target properties may be compiled and manually or automatically curated to exclude toxic compounds and compounds shown to promote disease or other harmful effects, for example cancer. Furthermore, compounds associated with normal metabolism of cells, e.g. dCTP, belonging to the superclass of nucleosides, nucleotides, and analogues and directly involved in deoxyribonucleic acid (DNA) synthesis may also be removed from the final curated list.

According to a fourth aspect of the invention, there is provided a computer system comprising: at least one processor; and memory. The memory stores computer readable instructions that, when executed by the at least one processor, causes the computer system to perform a method of any aspect of the invention.

The system may further comprise storage for storing interaction data and/or an interactome and/or a list of molecule(s) and/or biomolecule(s) and/or a biological cell(s) and/or a biological process(es) and/or a trained model.

According to an aspect of the invention, there is provided a computer-implemented method for predicting molecule properties, the method comprising: receiving a biological entity interaction graph; receiving an input molecule descriptor comprising at least a molecule interaction signal with a plurality of biological entities; computing input molecule interaction descriptor by applying at least one layer of parametric diffusion to input molecule interaction signal on the biological entity interaction graph;

using the input molecule interaction descriptor to predict the input molecule properties; outputting the predicted input molecule properties.

The biological entities may be one or more of the following: gene; protein; metabolite; pathway; super-pathway; gene ontology.

The interactions between biological entities may be one or more of the following: protein binding.

The predicted input molecule properties may be one or more of the following: efficiency against at least one disease type; efficiency against cancer phenotype; toxicity.

The input molecule descriptor may further include a chemical descriptor.

The chemical descriptor may be obtained by applying a graph neural network to the molecular graph of the input molecule.

The input molecule interaction signal may comprise the interaction of the input molecules with each of the biological entities in the biological entity interaction graph.

The interaction of the input molecules with each of the biological entities may comprise at least binding affinity.

The biological entity interaction graph may further include one or more of the following: edge features representing the interactions between pairs of biological entities; node features representing the biological entities.

Computing molecule interaction descriptor may comprise one or more of the following: applying diffusion kernel to the molecule interaction signal on the biological entity interaction graph interaction graph; applying at least one layer of graph convolutional neural network to the molecule interaction signal on the biological entity interaction graph.

The parametric diffusion may be one of the following: random walk with a fixed transition matrix; diffusion process dependent on node and edge features; graph attention diffusion; non-linear graph message passing.

Using the molecule interaction descriptor to predict the molecule properties may comprise applying at least a neural network to the molecule interaction descriptor.

Computing input molecule interaction descriptor may further comprise pooling on the biological entity interaction graph. Pooling may further comprise a hierarchy of graphs obtained from the input biological entity interaction graph. Pooling may be learnable. Pooling may be done according to biological entities belonging to higher-level structures, which may include pathways.

At least the parameters of parametric diffusion may be determined by a training procedure.

The training procedure may further comprises: receiving a training dataset of molecules, said dataset comprising for each molecule at least the molecule interaction signal the molecule groundtruth property tuning the parameters to optimize a loss function.

The training set may further include, for each molecule, the molecule chemical descriptor.

The loss function may be one of the following or a combination of one or more of the following: a distance between the predicted molecule properties and the groundtruth molecule properties; classification error

The training set may comprise positive examples of drugs efficient against a disease and negative examples of drugs inefficient against a disease, and the predicted molecule property is efficiency against disease.

### BRIEF DESCRIPTION OF THE DRAWINGS

Certain embodiments of the present invention will now be described, by way of example, with reference to the accompanying drawings, in which:

FIG. 1 is a schematic diagram of the workflow;

FIG. 2 illustrates relevant genes and pathways derived from machine leaning models for prediction of anti-cancer therapeutics tested in human trials. Individual node size corresponds to the relative discriminating capacity of a given gene-encoded protein and node color illustrates shared biological pathway functionality.

FIG. 3 illustrates hierarchical classification of the top 110 predicted cancer-beating molecules in food with anti-cancer drug likeness of >0.7; and

FIG. 4 illustrates the contained profiles of compounds within selective foods, which were highly likely to be effective in fighting cancer. Each node in the figure denotes a particular food item and node size in each case is proportional to the number of CBMs. The link between nodes reflects the pairwise correlation profile of CBMs in foods, thus the clusters of foods illustrate molecular commonality between them.

FIG. 5 is a schematic block diagram of a first computer system;

FIG. 6 is a schematic block diagram of a second computer system;

FIG. 7 is a schematic block diagram of a third computer system;

FIG. 8 is a is a process flow diagram of generating a list of biomolecules, biomolecule process(es) and/or biological cell(s) in the interactome that are affected by a given molecule;

FIG. 9 is a process flow diagram of generating a trained model; Figure to is a process flow diagram of validating anti-target molecules;

FIG. 11 is a process flow diagram of generating a prediction for an anti-disease effect of a molecule;

FIG. 12 is a table of cancer beating molecules in different foods; and

FIG. 13 is a table of a list of machine learning-predicted compounds in foods and their anticancer likeness.

### DETAILED DESCRIPTION

Recent data indicate that up-to 30-40% of cancers can be prevented by dietary and lifestyle measures alone. Herein, we introduce a unique network-based machine learning platform to identify putative food-based cancer-beating molecules. These have been identified through their molecular biological network commonality with clinically approved anti-cancer therapies. A machine-learning algorithm of random walks on graphs (operating within the supercomputing DreamLab platform) was used to simulate drug actions on human interactome networks to obtain genome-wide activity profiles of 1962 approved drugs (199 of which were classified as “anti-cancer” with their primary indications). A supervised approach was employed to predict cancer-beating molecules using these ‘learned’ interactome activity profiles. The validated model performance predicted anti-cancer therapeutics with classification accuracy of 84-90%. A comprehensive database of 7962 bioactive molecules within foods was fed into the model, which predicted 110 cancer-beating molecules (defined by anti-cancer drug likeness threshold of >70%) with expected capacity comparable to clinically approved anti-cancer drugs from a variety of chemical classes including flavonoids, terpenoids, and polyphenols. This in turn was used to construct a ‘food map’ with anti-cancer potential of each ingredient defined by the number of cancer-beating molecules found therein. Our analysis underpins the design of next-generation cancer preventative and therapeutic nutrition strategies.

### INTRODUCTION

The human diet contains thousands of bioactive molecules which modulate a variety of metabolic and signalling processes, drug actions, and interactions with gut microbiota in health and disease. Investigating the influence of a single biochemical food constituent takes months to years of experimental research. Moreover, current approaches to identify active compounds within food that influence health are incapable of taking into consideration the myriad of complicating factors such as where the food comes from, how it has been cultivated, stored, processed and prepared, not to mention cooking parameters and the effect of ingredient combinations. Given the vast molecular space, predictive identification of bioactive compounds for tailored nutritional strategies using current experimental research methods is therefore not feasible. However, recent advances in AI technologies coupled with the explosive growth of large-scale multi-source (“-omics”) data on food, drugs and diseases offers a unique opportunity to identify molecules within foods to potentially prevent and/or fight disease phenotypes. These studies have identified molecules within foods based on either structural similarity or the similarity of individual gene-encoding protein targets to those of approved therapeutics. However, even minor change in the chemical structure of a molecule can lead to drastically different biological outcomes, and complex diseases, such as cancer, cannot be explained by deregulated activity of individual genes/proteins. Several recent computational studies have attempted to leverage “-omics” data to extract insights on positive and/or adverse interactions between foods, drugs and disease. Zheng et al. used publicly available gene expression and interactome data of cell cultures and animal models to identify drugs and diets anti-correlated with disease gene expression phenotypes. Due to the small size of existing diet-induced gene expression datasets, this correlation-driven analysis was restricted to a very limited number of foods. Nevertheless, intriguing diet-disease associations have been identified through this approach. A combined chemo-informatics and text mining strategy was applied to several million PubMed abstracts to define health-promoting or detrimental associations between the molecular constituents of plant-based foods and disease phenotypes. This strategy was subsequently extended to identify food components interfering with drug metabolizing enzymes (“pharmacokinetics”) or interacting with drug targets (“pharmacodynamics”). Although of great promise, the automated relation extraction systems based on natural language processing (NLP) have thus far been tested on a very small subset (<200) of somewhat subjectively annotated abstracts. As we highlighted recently, their application at the scale of multi-million article databases such as PubMed warrants extensive validation of the rate of false discoveries and extraction of supporting evidence to build trust in the computer-derived associations. Nevertheless, these developments have been instrumental to the compilation of “-omics” food databases and public repositories such as FooDB, FlavorDB and NutriChem.

Complex diseases such as cancer cannot be explained by single gene defects but rather involves a breakdown of various molecular functions mediated through a set of molecular interactions (“networks”). The diversity of the resulting cancer molecular phenotypes makes it very difficult to identify specific molecular targets for cancer prevention or treatment. We hypothesize that an effective cancer preventative or therapeutic intervention should target multiple biochemical pathways implicated in carcinogenesis such as inflammation, cell proliferation, cell cycle, apoptosis and angiogenesis. In line with this hypothesis, we have tailored a machine-learning based strategy that predicts CBMs based on “learned” molecular networks targeted by clinically validated anti-cancer therapies. Our strategy includes the combined use of unsupervised learning on graphs to simulate the downstream influence of therapeutics on human proteome networks (from “sparse” protein target datasets) followed by supervised learning to identify predictive (sub-)networks for CBMs. Model performance was assessed using a 10-fold cross-validation strategy, which confirmed accurate prediction of anti-cancer therapeutics. A comprehensive database of 7692 bioactive molecules within foods was fed into the model to predict ˜110 CBMs, resulting in a compiled list of hyperfoods exhibiting the largest number of potential CBMs (ACL>0.7). Furthermore, the developed approach can be easily extrapolated in the future to cover other types of diseases (e.g. diabetes) and health issues to provide a comprehensive multi-faceted picture of health-promoting food molecules and optimize existing cooking recipes for the maximally positive health impact. We envisage that this first list of “cancer-beating” foods will serve as one of the pillars in the foundation for the future of gastronomic medicine and should aid the creation of personalized “food passports” to provide nutritious, tailored and therapeutically functional foods for the population. However, significant future work will be required to validate and quantify the therapeutic effects of these proposed hyperfoods as well as optimize cultivation, storage, processing and cooking parameters of their ingredients.

### Results and Discussion

### Network-Based Machine-Learning Strategy for Drug and Food Repositioning.

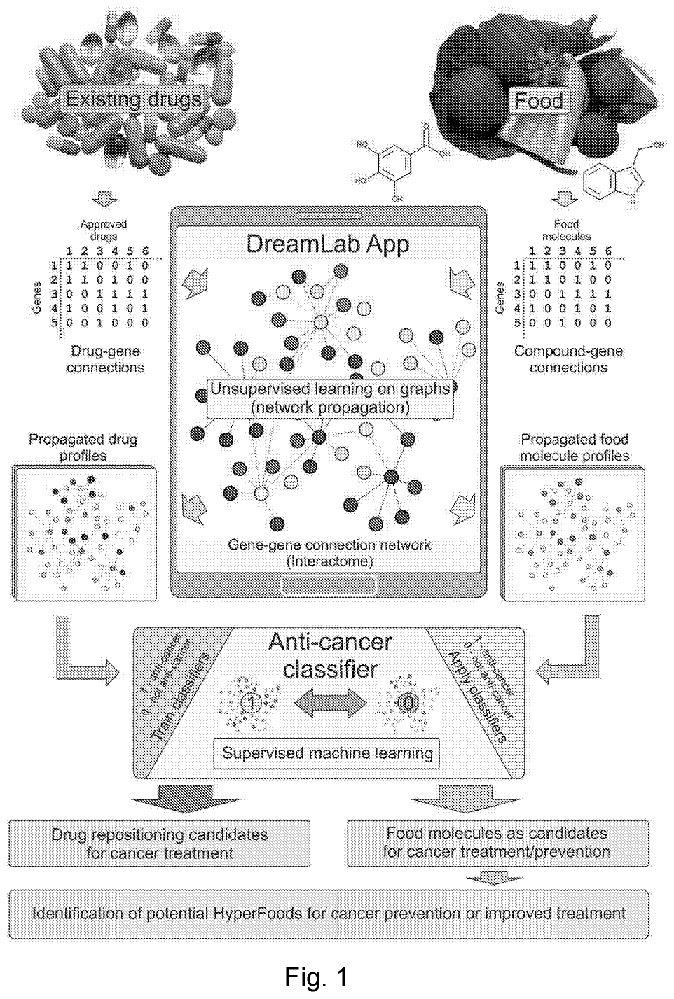

The work presented herein exploits publicly available data on molecule to gene-encoded protein interactions as well as protein-protein interaction data. In brief, the sparse data of interactions between drugs and their protein/gene targets are initially mapped on large-scale interactome networks—a whole set of protein-to-protein interactions in humans (here and further due to the specifics of the existing interaction datasets, “gene” and “protein” terms can be used interchangeably). Most drugs exert their biomedical and functional activity by binding to a specific subset of proteins. Proteins rarely function in isolation but rather operate as part of highly interconnected networks. Taking this into account, we have tailored random walks on graphs with restarts (controlled by a single network diffusion parameter “c”) to simulate the perturbation of individual drugs on human proteome networks using aggregated datasets of their targeted proteins. Similar network-based propagation approaches have been recently compared favourably to predict drug-target interactions, and evaluate network perturbations caused by cancer mutations for improved patient stratification. This network diffusion transforms a short list of proteins targeted by a given molecule/drug into a genome-wide profile of gene scores based on their network proximity to target candidates. Using the genome-wide profiles of drugs, the supervised machine-learning strategy (“maximum margin criterion” and support vector machines, in this case) is trained to accurately classify “anti-cancer” (vs “other”) properties of molecules. The best obtained models were used to predict the probability of a given existing approved drug to exhibit anti-cancer properties. After validation of the predictive capacity of the model for anti-cancer drug repositioning, the same machine learning strategy was applied to predict various cancer-beating molecules within foods (see FIG. 1). It should be noted that there are various methodologies for drug repositioning such as molecular structural commonality, molecular target similarity as well as shared genetic or phenotypic (e.g. side effect profile) influence. However, these approaches mandate additional data sets (such as gene-expression data, proteomics, metabolomics or phenotypic effect data) for model building. In the search for food-based cancer beating molecules, these data are very limited.

### Benchmarking and Optimization of Machine Learning Strategy.

Among the machine learning methods tried, MMC (maximum margin criteria) and SVM with linear kernel showed comparable performance and relatively good processing speed (including parameter optimization, model training and prediction on 10-fold cross-validation). Radial kernel SVM did not exceed the performance of the linear methods and at the same time required much longer processing time (the best radial kernel SVM F1-score achieved is of 0.85 vs 0.86 for linear kernel SVM). Furthermore, the optimal gamma parameter for the radial SVMs tends to be very low (˜10⁻⁷), effectively making them similar to the linear kernel SVMs. We have also explored 2 neural network classifiers and 2 regularized LASSO/Elastic Net logistic classifiers to see whether they bring any improvement in the classification accuracy. For the best performing type of interactome and settings of random walk on graphs, these more advanced approaches resulted in prediction accuracies comparable to linear SVM and MMC (see Supplementary Information Appendix M1 below). This is well known in genomics studies involving a small number of examples and a large number of features, where the linear classifiers are preferred because of their transparency and biological interpretability. As a result, the major focus was made on linear kernel SVM and MMC methods for the final round of optimization. The best F-score achievable was of 0.86 with linear kernel SVM with 84% correct anti-cancer predictions and 90% correct non-anticancer predictions (see Supplementary Information Dataset S1 in Veselkov et al., “HyperFoods: Machine intelligent mapping of cancer-beating molecules in foods”, Scientific Reports, 2019, 9:9237). Re-running the optimization multiple times for the same settings showed consistent performance (maximum 1-2% difference). Based on these results, it was decided to select the top 700 models (F-score>=0.84) for anti-cancer likeness prediction from models based on linear kernel SVM and MMC for existing approved drugs (Supplementary Information Dataset S2 in Veselkov et al. 2019) and food compounds (Supplementary Information Dataset S3 in Veselkov et al. 2019). Interestingly, log-transformation of the input propagated profiles was systematically shown to increase performance of the classifiers. This is likely because some individual isolated genes, which do not propagate and thus stay with a very high perturbation level would have lesser effect on the overall profile in log-space. At the same time “c” parameter of the random walker and different matching settings between compounds and genes had less pronounced effects. Gene-gene connection thresholds were also not strongly influential except in the case of BioPlex interactome. This is likely because connections provided by STRING tend to include a wide range of knowledge sources giving a more representative and complete graph of gene-gene (or protein-protein) interactions and the sheer number of connections can compensate for the larger values of “c” and higher thresholds used. We have also evaluated individual gene influence on the final classification, i.e. gene importance, by finding the correlation between the gene levels and the prediction outcomes for the optimized model. The full table of averaged importance predictions for the top selected 700 models is provided as Supplementary Information Dataset S4 in Veselkov et al. 2019. As expected, the top-rated genes are involved in cell proliferation control and their mutations are often associated with cancer. This provides transparency to the machine learning based prediction of anti-cancer properties of the drugs.

### Pathway Analytics and Differential Interactome.

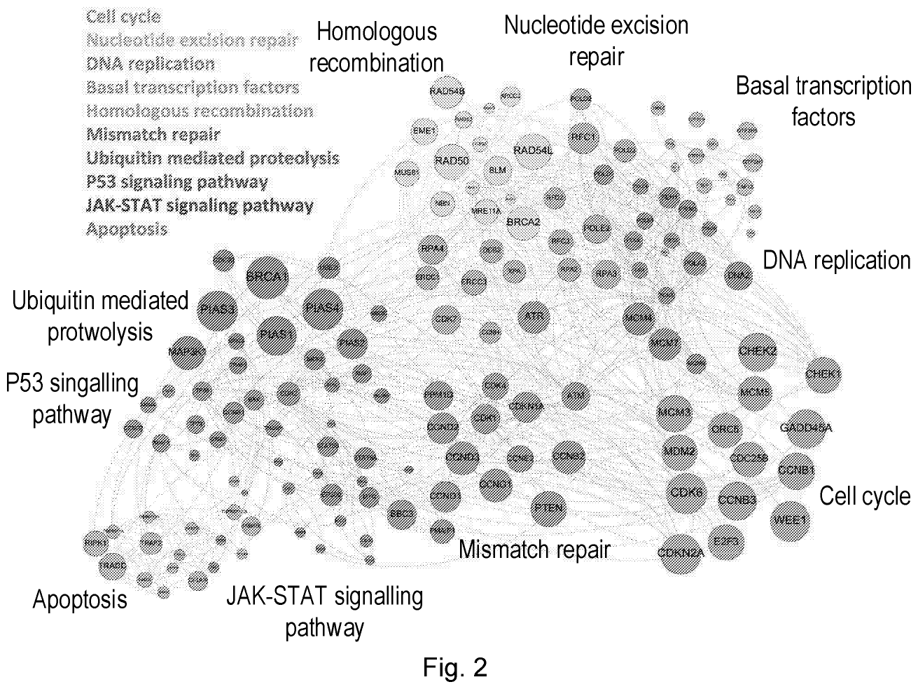

A list of the most influential genes/proteins for predicting anti-cancer therapeutics derived from network-based machine learning was subjected to pathway analytics using gene-set enrichment (Supplementary Information Dataset S4 in Veselkov et al. 2019). Among the top 25 impacted pathways were cell cycle, DNA replication, apoptosis, p-53 signalling, JAK-STAT signalling and mismatch repair as well as various cancer-specific pathways. It adds to the biological plausibility of the modelling approach used here that the pathways identified as key drivers are those consistently implicated in cancer development and progression. In FIG. 2, relevant discriminating genes and their corresponding impacted pathways are presented. Here, individual node size corresponds to the relative discriminating capacity of a given gene-encoded protein and node color illustrates shared biological pathway functionality. Increasingly, it is understood that the mechanistic bases for cancer survival, dissemination and therapeutic resistance are manifold and involve multiple biochemical pathways. Most machine-learning derived pathways in our analysis have been suggested as targets for cancer prevention or therapeutic interventions 30-32. Therefore, the “ideal” anti-cancer agent should be capable of disrupting multiple pro-tumorigenic biochemical processes. The machine learning approach presented here highlights the biological pathways influenced by currently utilized anti-cancer therapeutics, and thus permits in parallel a targeted search for unique agents, in this case bioactive compounds with foods, with the potential to impact on multiple pathways simultaneously.

Drug Repositioning in Cancer Using Interactomics.

The full prediction summary is presented in Supplementary Information Dataset S2 in Veselkov et al. 2019. As expected most compounds currently in use as cancer therapeutics demonstrated strong anti-cancer probability. Interestingly, several compounds which are not conventionally used in cancer treatment demonstrated very high anti-cancer likeness (ACL). The available literature on these compounds was further interrogated to understand the mechanistic basis for the potential anticancer effect(s) of these agents. For example, quinolone-derivative rosoxacin and quinoline-based clioquinol primary act as anti-microbial and anti-fungal agents, respectively. However, the analysis presented here indicates a potential direct role for these therapeutics in cancer. The quinolone antibiotics were shown to have a significant inhibiting potency against eukaryotic topoisomerase-II resulting in cytotoxicity of various cancer cell types. This group of compounds can be explored in comparison to human topoisomerase-II inhibiting anti-tumor drugs such as doxorubicin and etoposide. Clioquinol is a chelator of zinc, copper and iron which are known to be involved in both carcinogenesis and angiogenesis. The anti-neoplastic activity of clioquinol is thought to be through several potential mechanisms including NF-kB apoptosis induction, mTOR signaling and inhibition of lysosome. Although of great promise its role in cancer therapy remains largely unexplored in clinical settings. The anti-diabetic drugs such as metformin and chromium picolinate, also emerged as potential candidates for anti-cancer drug repositioning from this evaluation. The molecular mechanisms responsible for this association remain uncertain, however both agents are used to alleviate insulin resistance through modulation of the insulin signaling cascade, and a number of studies have shown that chromium specifically alters proximal insulin signaling and directly effects insulin receptor phosphorylation and kinase activity. The downstream consequences of therapy with both metformin and chromium is the reduction in insulin and insulin-like growth factor levels, which in turn is understood to inhibit several key processes within the mTOR signaling pathway, which is a central molecular driver of a variety of cancers. Correspondingly a strong association has been shown on pooled analysis between metformin usage and incidence of cancer in type II diabetics. By contrast, the chromium picolinate might act as a double “edged sword” due to its capacity to interfere with DNA leading to structural genetic lesions and thereby promoting carcinogenesis. This example highlights the limitation of our approach to identify molecules that interact with relevant carcinogenetic processes irrespective of the nature of the interaction (i.e. inhibition or stimulation). Identifying the nature of molecular interactions would require additional datasets such as gene expression or proteomics but these are not generally available in the case of food-based molecules.

### Prediction of Cancer-Beating Molecules in Foods.

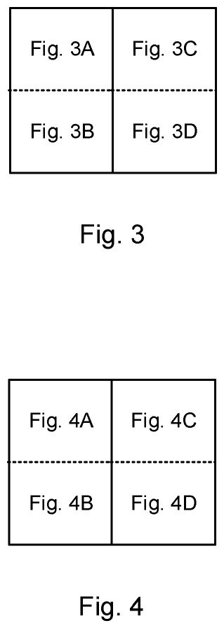

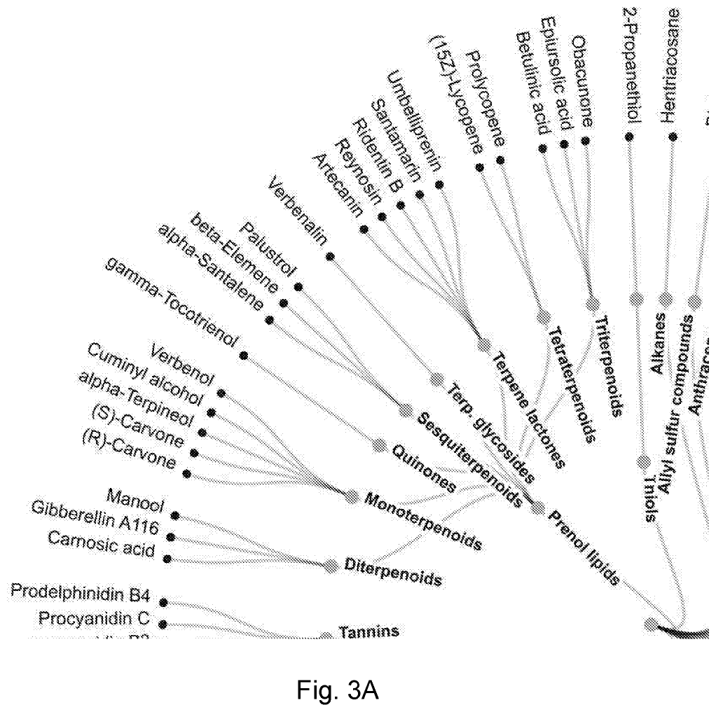

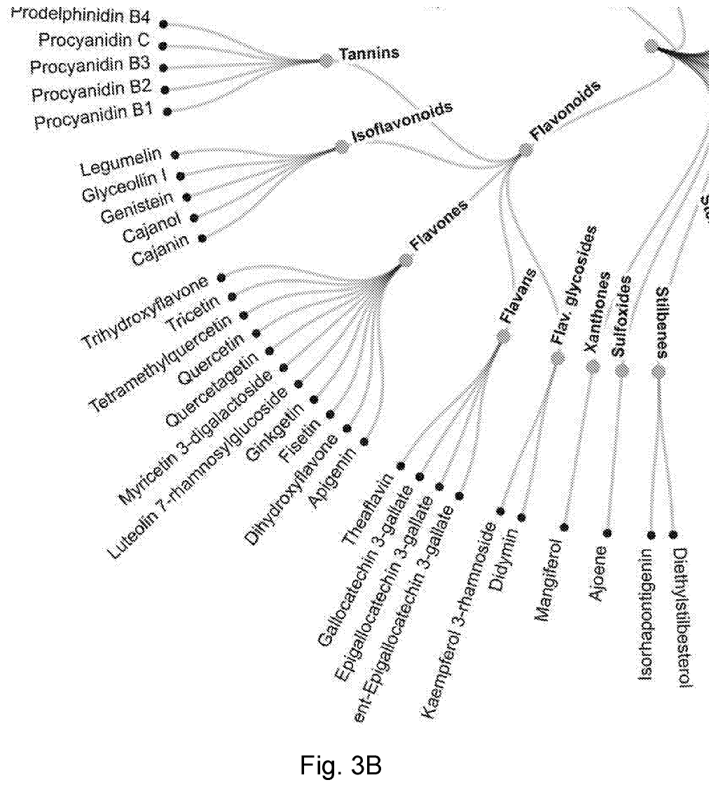

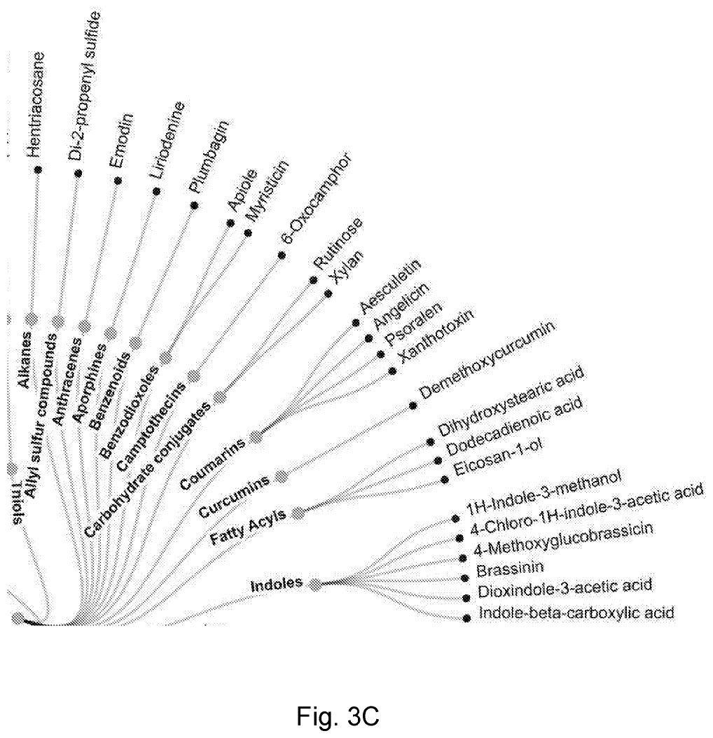

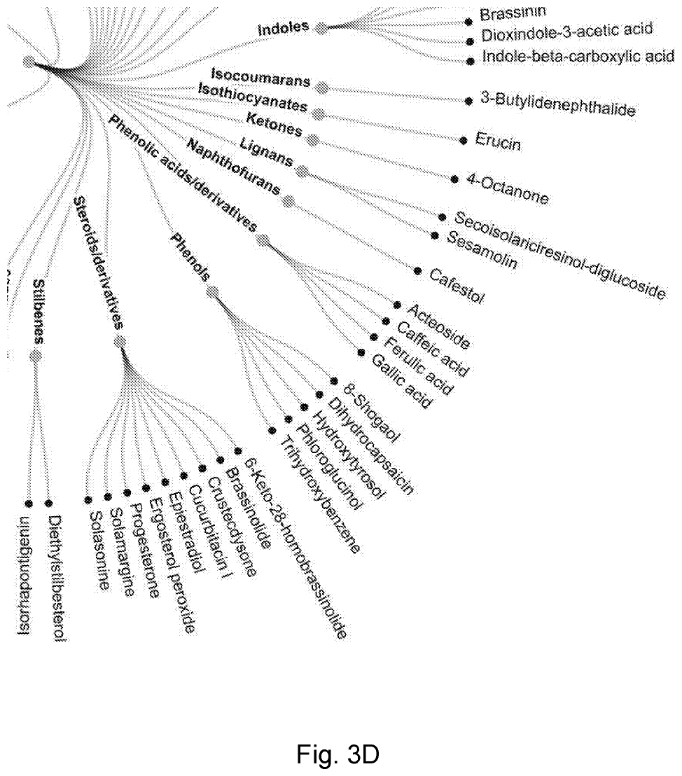

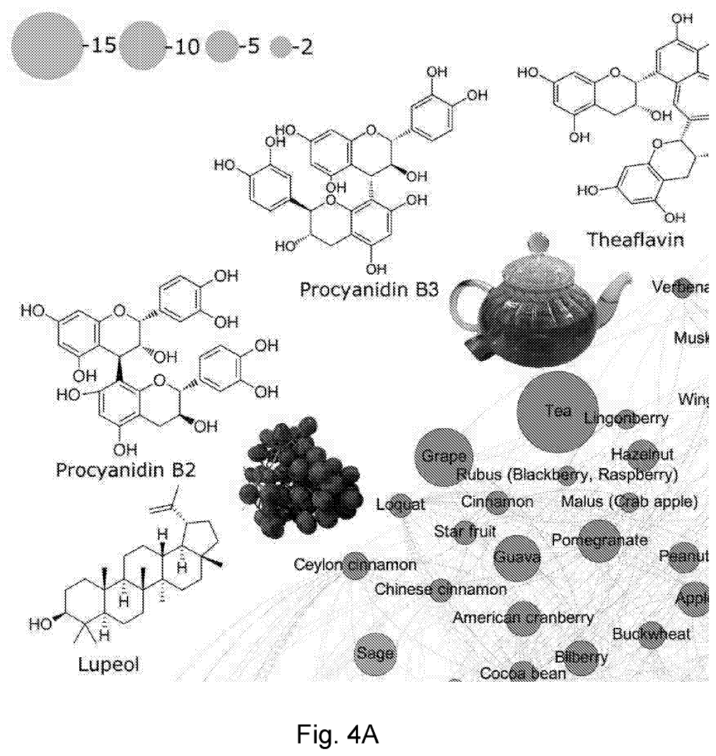

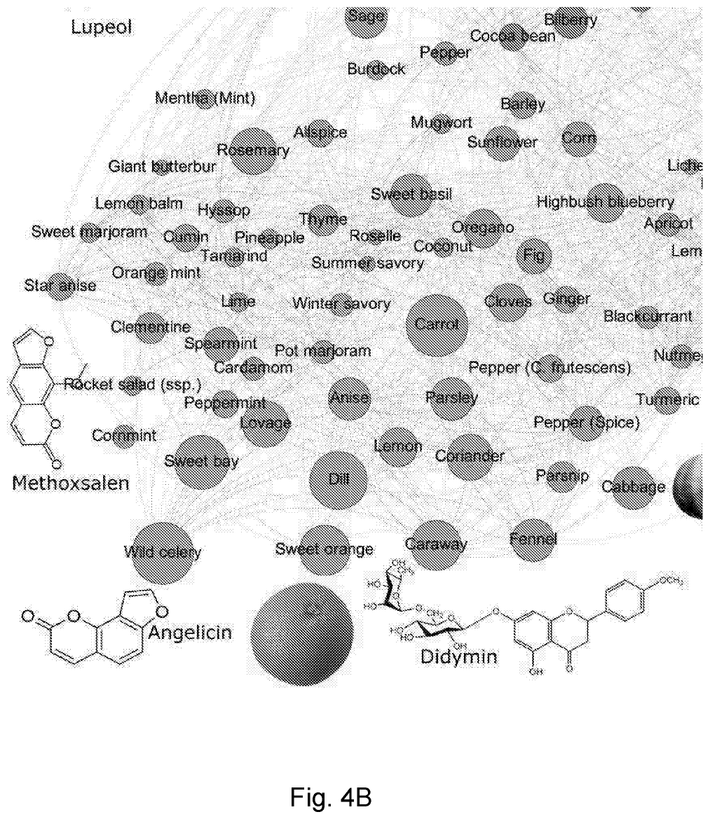

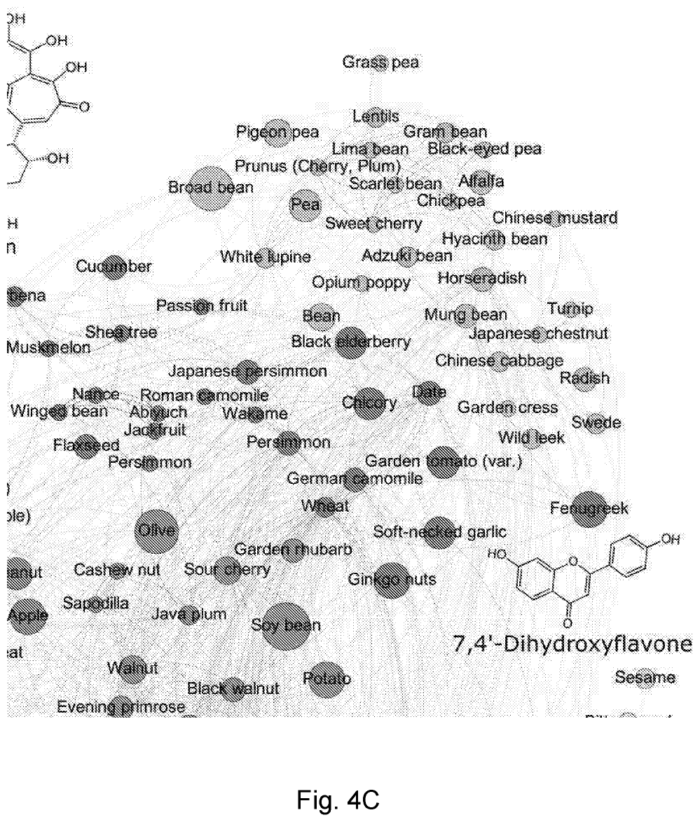

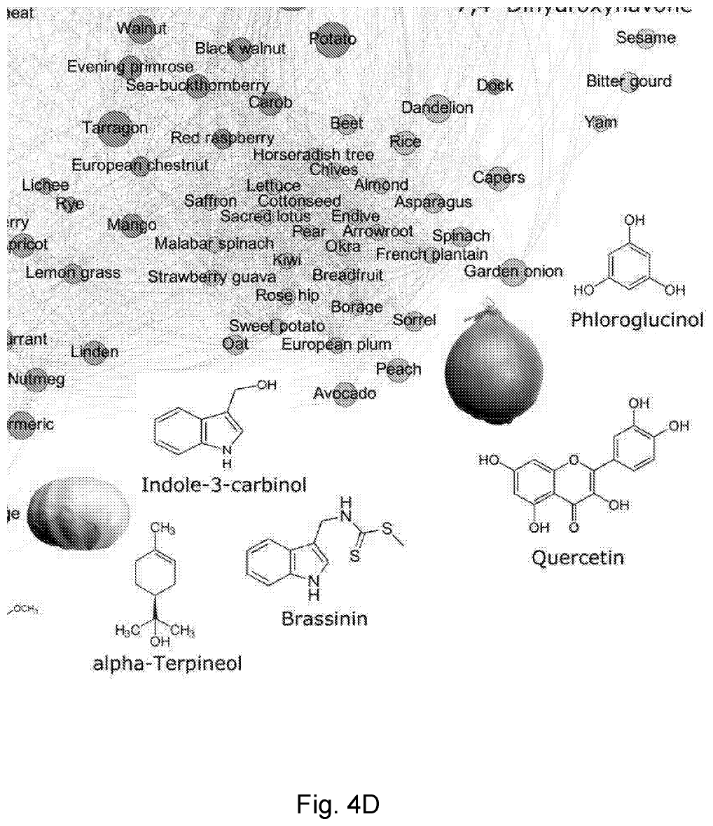

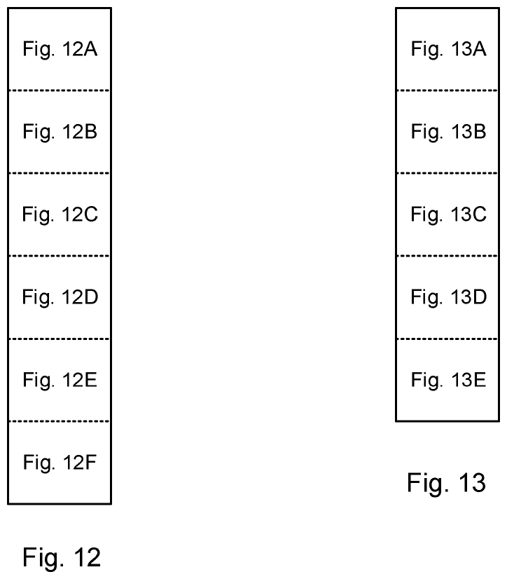

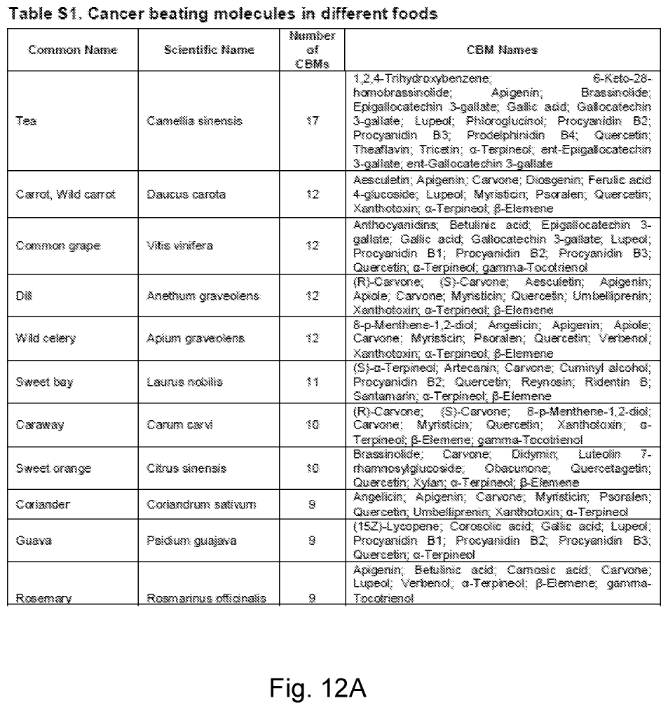

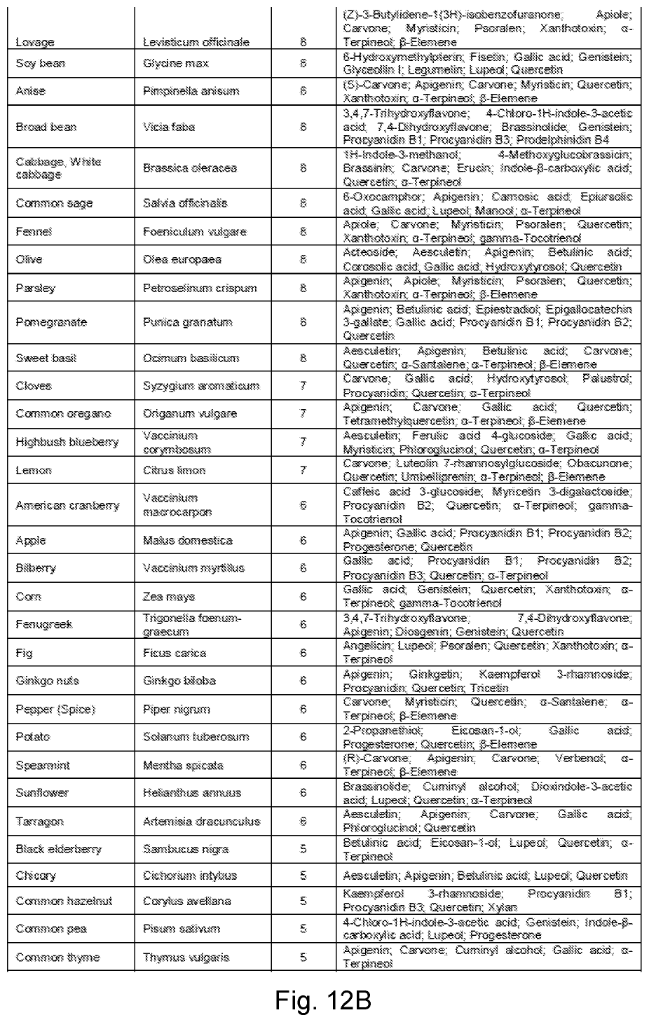

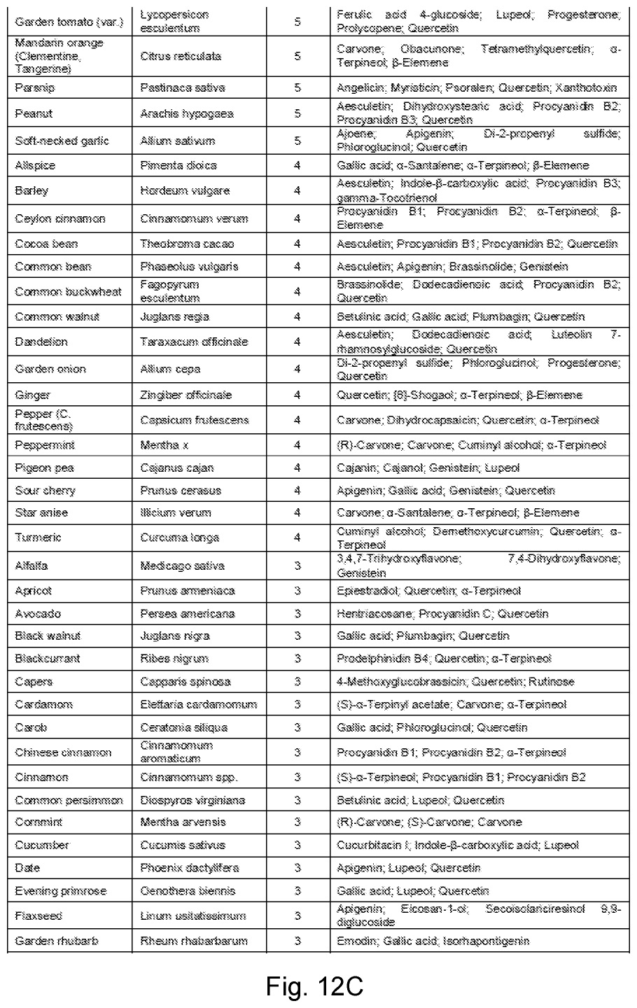

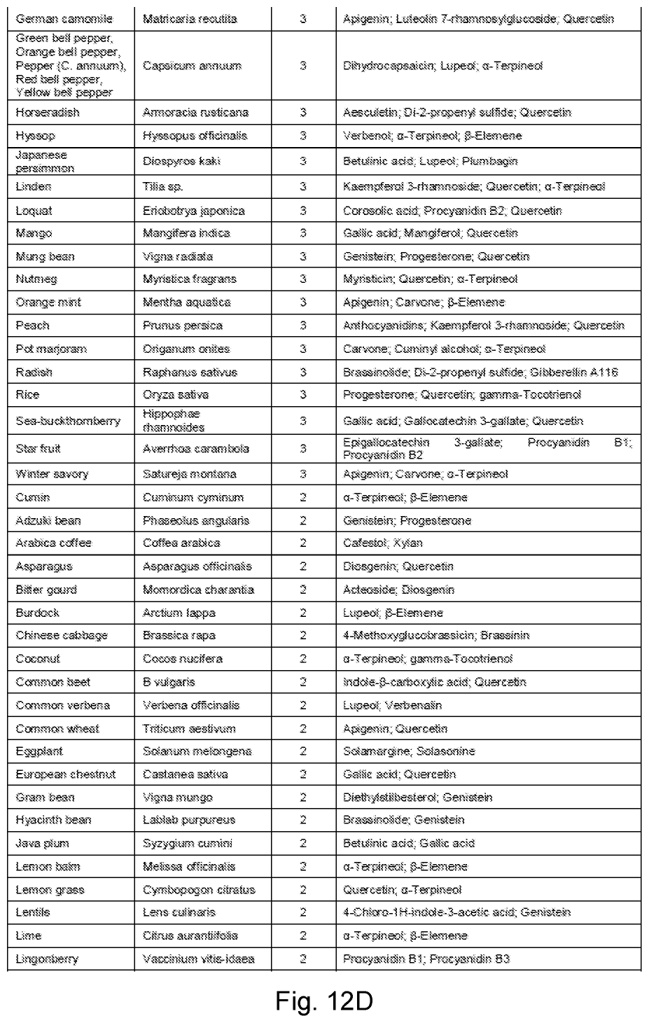

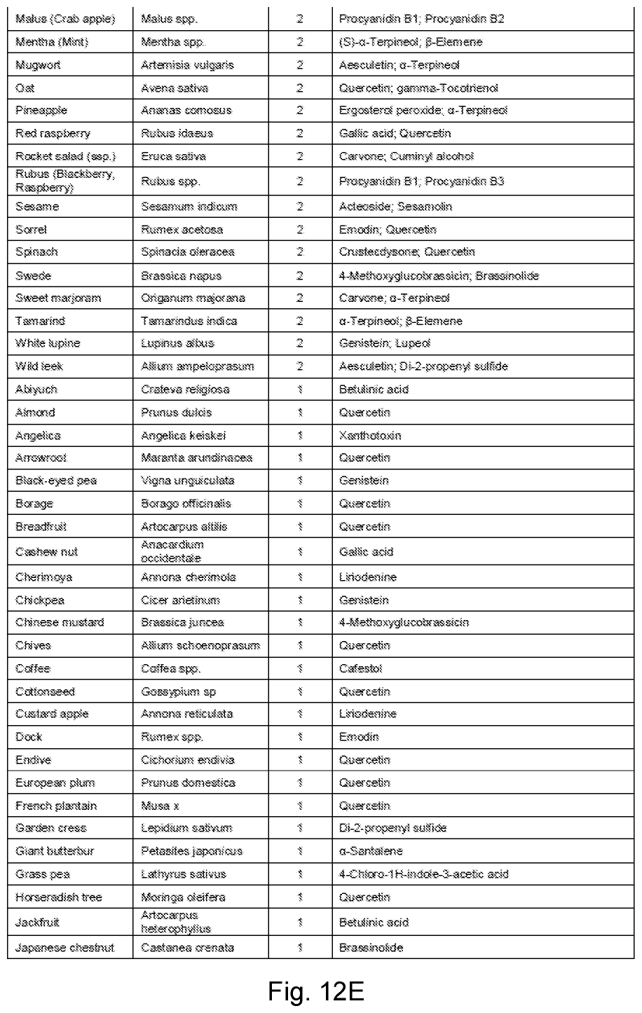

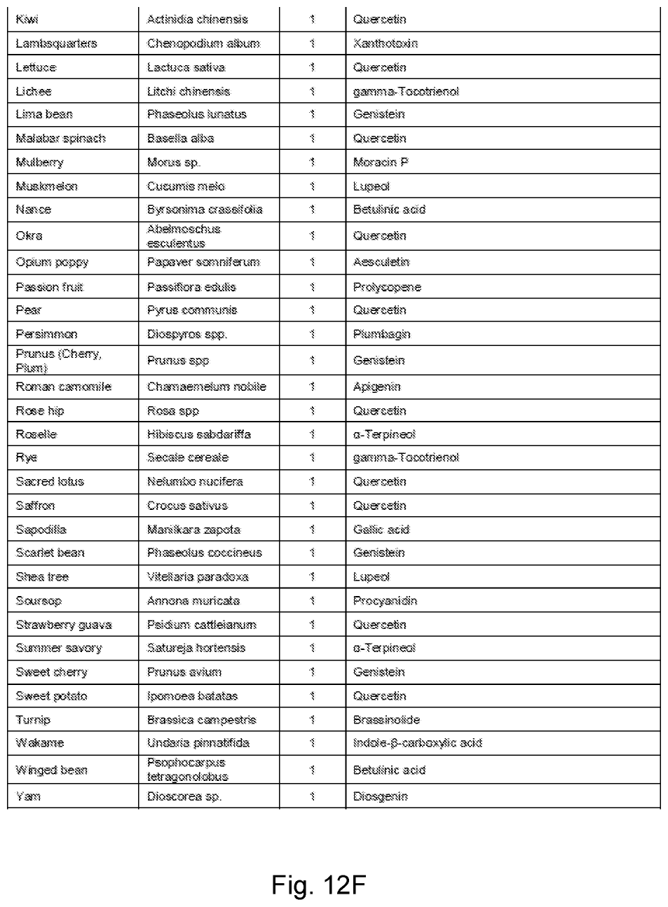

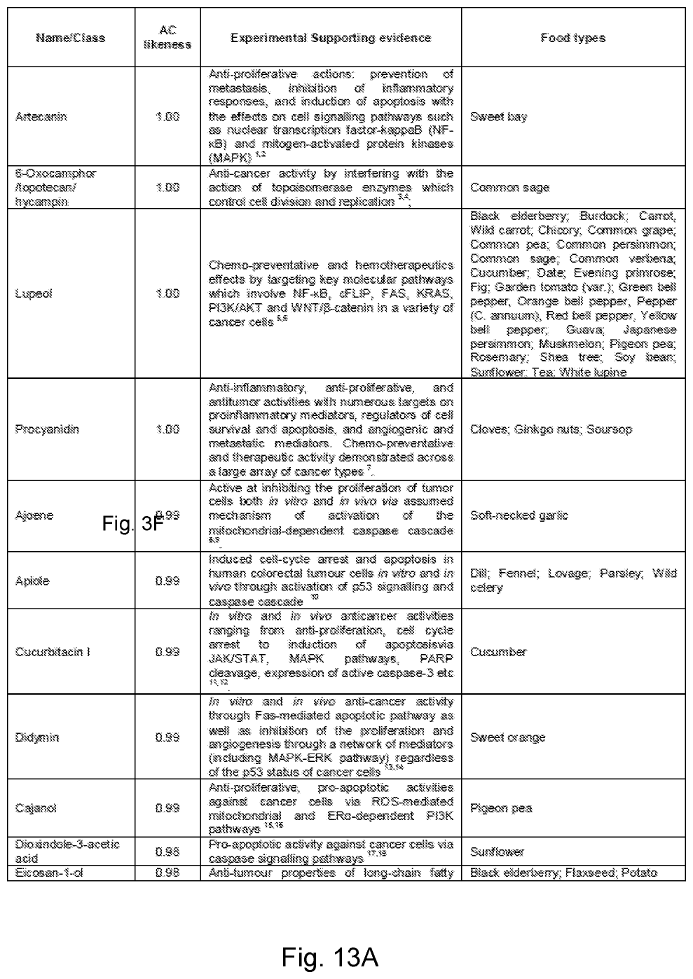

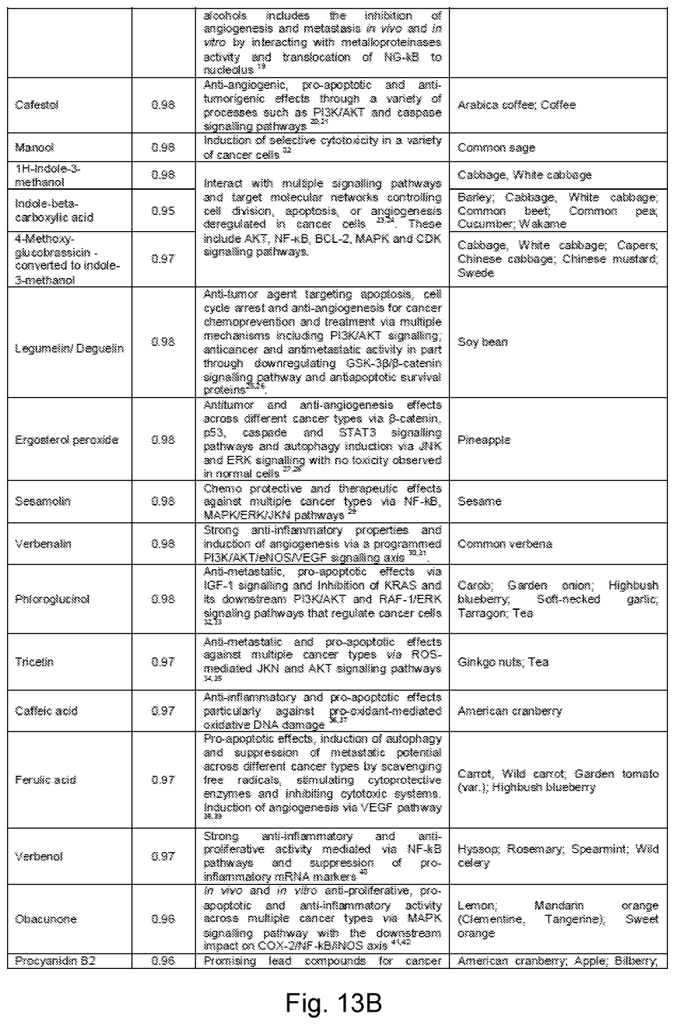

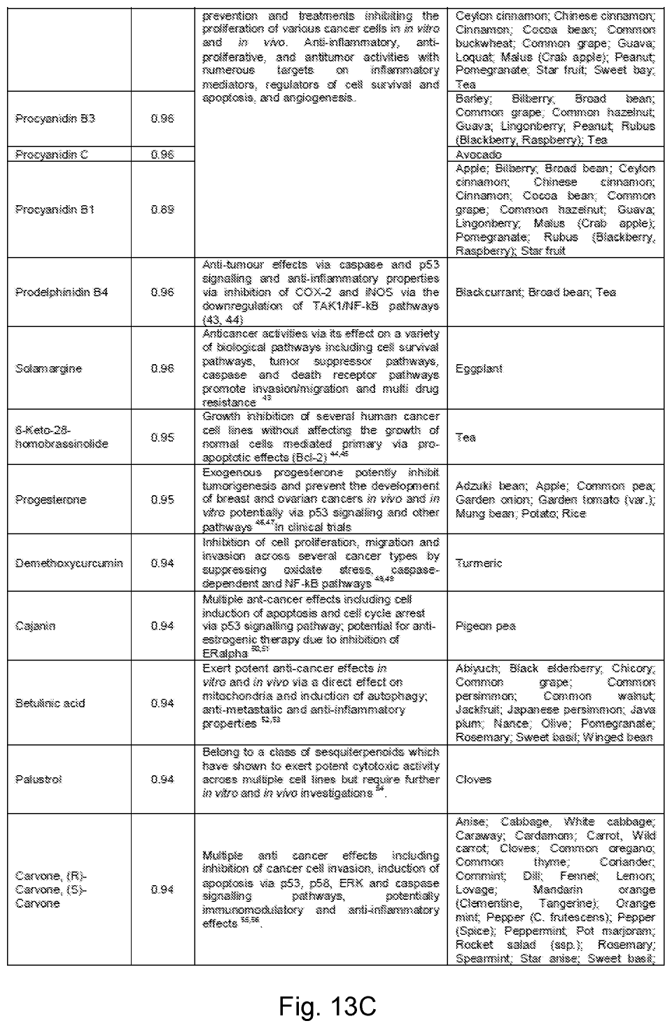

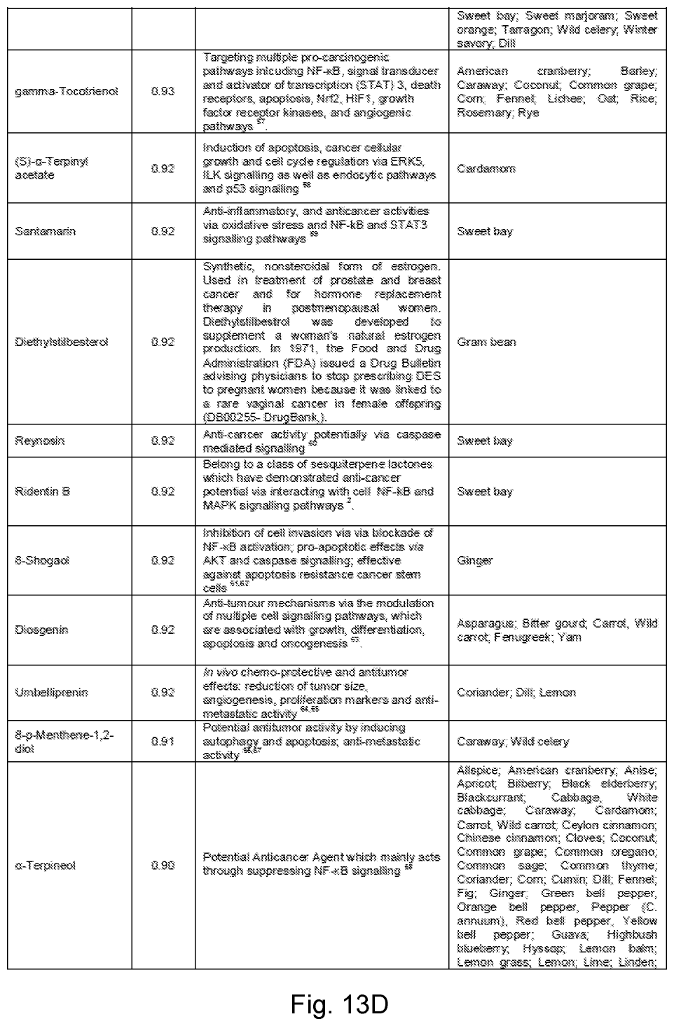

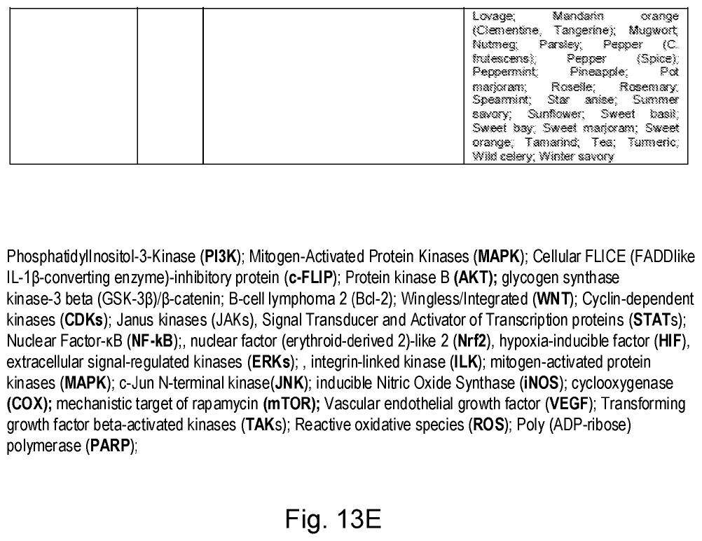

From all small molecules approved for anti-cancer therapies, almost half are derived from natural products. These drugs are generally more tolerated and less toxic to normal cells. The methodology outlined above was next applied to predicting the anti-cancer likeness of ˜7692 bioactive compounds across various food categories. Here a comprehensive view of drug-like molecules in food is provided, unlike most studies in the literature to date which have tended to focus on a single compound or a single food type. Approximately 110 molecules from different chemical classes (see FIG. 3), including terpenoids, isoflavonoids, flavonoids, poly-phenols and brosso-steroids were identified and mapped according to their food sources using multiple experimental databases. A complete list of food molecules ranked by proxy according to anti-cancer drug likeness of >0.1 is provided in Supplementary Information Dataset S3 in Veselkov et al. 2019. Using the unsupervised learning random walk on graphs, we have propagated the influence of the most promising molecules on human interactome networks and identified their impacted molecular pathways (for detailed analysis see Supplementary Information Dataset S3 in Veselkov et al. 2019 and Supplementary Information Dataset S5 in Veselkov et al. 2019 only for compounds with ACL>0.7). Supplementary Information Appendix Table S1 in Veselkov et al. 2019, and FIG. 12 summarizes a list of cancer-beating compounds identified in the present study with high ACL>0.7 and their associated food sources. Furthermore, we have conducted a comprehensive review of the available literature on the top anti-cancer drug like molecules (with ACL>0.9) and their putative molecular mechanisms of anti-cancer actions (Supplementary Information Appendix Table S2 and FIG. 13). Both computational analysis and experimental data from literature show that the pathways and mechanisms responsible for these anti-cancer properties cover the breadth of our current understanding of the multi-step process of carcinogenesis. These include anti-inflammatory, pro-apoptotic effects, potent antioxidant activity and scavenging free radicals; regulation of gene expression in cell proliferation, cell differentiation, oncogenes, and tumor suppressor genes; modulation of enzyme activities in detoxification, oxidation, regulation of hormone metabolism; and antibacterial and antiviral effects. For example, 3-indole-carbinol, which is found abundantly in members of the Brassica oleracea family of vegetables (including cabbage, broccoli and brussel sprout) appears to be one of the most strongly anti-cancer-like molecules. This bioactive compound has been shown to target multiple aspects of cancer cell cycle regulation and survival, including caspase activation, oestrogen metabolism and receptor signalling and endoplasmic reticulum function (see Supplementary Information Appendix Table S2 in Veselkov et al. 2019 and FIG. 13 and reference therein). Other prominent examples include dydamin, which is a flavonoid glycoside found in citrus fruits and apigenin, which is particularly abundant in coriander, parsley and dill. Both are understood to influence apoptotic pathways as well as cell cycle arrest mechanisms and are believed to suppress cancer cell migration and invasion (see Supplementary Information Appendix Table S2 in Veselkov et al. 2019 and FIG. 13 and reference therein). FIG. 4 provides a visual summary of CBMs associated with strong anti-cancer likeness. Each node in the figure denotes a particular food item and node size in each case is proportional to the number of CBMs. The link between nodes reflects the pairwise correlation profile of CBMs in foods, thus the clusters of foods seen in FIG. 4 illustrate molecular commonality between them. The foods that show greatest diversity in CBMs include tea, grape, carrot, coriander, sweet orange, dill, cabbage and wild celery.

### Food Map and Phytochemical Synergy.

The potential of food sources to exert their preventative or therapeutic capacity depends upon the bioavailability and diversity of disease-beating molecular compounds contained therein. A key limitation in regards to the existing literature on food-based compounds is the largely one-dimensional view that is commonly taken, with studies tending to focus on specific molecular components in isolation, for example anti-oxidants 40. It is accepted that regular consumption of fruits and vegetables can reduce the risk of carcinogenesis. However, when antiproliferative agents acting in isolation have been subjected to clinical trial evaluation they do not appear to consistently confer the same level of benefit. The point is simply illustrated in the case of the apple; apple extracts contain bioactive compounds that have been shown to inhibit tumor cell growth in vitro. However, interestingly phytochemicals in apples with the peel preserved inhibit colon cancer cell proliferation by 43%, whereas this effect was found to be reduced to 29% when apple without peel was tested. From these observations it is therefore clear that the successful implementation of food-based approaches in the fight against complex diseases such as cancer will rely on a consortium of biologically active substances, such as those present in whole fruits and vegetables, in order to increase the chances of success. The anti-cancer properties of a given food will thus be determined by (1) the additive, antagonistic and synergistic actions of their individual components and (2) the way in which these simultaneously modulate different intracellular oncogenic pathways. Both of these conditions are fulfilled in the case of tea for example, which we found to strongly exhibit anti-cancer drug-like properties compared with other food ingredients. Tea is a rich source of anti-cancer molecules from catechins (epigallocatechingallate), terpenoids (lupeol) and tannins (procyanidin) and, three of which exert strong and complementary anti-cancer effects, by protecting reactive oxidative species induced DNA damage, suppressing inflammation and inducing apoptosis and cancer cell cycle arrest, respectively. Correspondingly, several recent meta-analyses demonstrated that the consumption of green tea demonstrated delayed cancer onset, lower rates of cancer recurrence after treatment, and increased rates of long-term cancer remission. Other examples include citrus fruits such as sweet orange, which contains dydimin (citrus flavonoid), obacunone (limonoid glucose) and β-elemene with strong anti-oxidant, pro-apoptotic and chemosensitization effects, respectively. The latter have strong effects particularly against drug-resistant and complex malignancies across different types of cancers. The inverse associations between citrus fruit intake and incidence of different types of cancers were confirmed by meta-analysis of multiple case-control and prospective observational studies. With this understanding we have constructed the anti-cancer drug-like molecular profiles comprised of over 250 different food sources (see FIG. 4 and Supplementary Information Appendix Table S1 in Veselkov et al. 2019 and FIG. 12).

### CONCLUSIONS

Using a network-based machine learning method, we have shown that plant-based foods such as tea, carrot, celery, orange, grape, coriander, cabbage and dill contain the largest number of molecules with high anti-cancer likeness through exerting influence on molecular networks in a similar fashion to existing therapeutics. Our large scale computational analysis further demonstrates more cancer-beating potential of certain foods calling for more tailored nutritional strategies. However, it is also important to acknowledge the limitations of the proposed methodology; firstly, concentrations of bioactive molecules are not taken into account and it is unclear they would be present in sufficient enough concentration to exert their beneficial biological activity. Furthermore, the proposed methodology only accounts for interactions between bioactive food compounds and cancer-related molecular networks, without explicit regard for directionality of these relationships. In addition, the methods described here do not take into account specific cancer molecular phenotypic characteristics. Finally, drug-food interactions have not been evaluated, and it is not clear whether these will lead to synergistic or antagonistic effects where they act on common molecular networks (pharmacodynamics), or whether this combination will disrupt drug metabolism itself (pharmacokinetics). Nevertheless, food represents the single biggest modifiable aspect of an individual's health and the machine learning strategy described here is a first step in realizing the potential role for “smart” nutritional programmes in the prevention and treatment of cancer. The outlined methodology is not restricted to cancer and will be applicable to other health conditions. Moreover, it will pave the way to the future of hyperfoods and gastronomic medicine, encouraging the introduction of personalized “food passports” to provide nutritious, tailored and therapeutically functional foods for every individual in order to benefit the wider population.

### Methods

### DRUGS/DreamLab Mobile Cloud Supercomputing.

The methodology and results presented in this manuscript were generated within the framework of the DRUGS project (Drug Repositioning Using Grids of Smartphones) run by Imperial College London in collaboration with Vodafone Foundation. The project has benefitted from the use of smartphone-based cloud supercomputing utilizing the DreamLab App. In brief, DreamLab allows a user to donate their idle smartphone computing power for use in large-scale computational tasks. With tens-to-hundreds of thousands of smartphones united into a cloud-based computational grid, one can split computational tasks into small chunks and run them in parallel. With enough contributors, the resulting performance compares to modern high performance computing clusters.

The DRUGS project uses publicly available data about gene-gene, protein-protein, drug-gene and drug-protein interactions to model systemic effects of the drugs and disease causing mutations. This allows to find promising candidates for drug repositioning and gene-tailored selection of drug combinations for treatment of different cancer types. Due to a massive number of potential combinations of drugs, cancer mutations and parameter settings, this project requires distributed computing to achieve viable speed and it fits perfectly within the specifications of the DreamLab architecture (high CPU usage, small memory footprint, no data exchange between jobs, small volumes of data transfer). The results presented in this manuscript are based on the initial data obtained within the DRUGS project with the aid of the DreamLab cloud computing platform, i.e. full propagated profiles of interactome impacts of different individual drugs and food compounds obtained for a wide range of settings. The predicted anti-cancer candidates are identified based only on the similarity of their full profiles to the known approved and clinically used anticancer drugs, which is established via machine learning approaches. Combinatorial analysis and gene-tailoring for personalized treatment recommendations are currently “work-in-progress” and fall outside of the scope of the present study.

### Aggregation of Molecular Data Sets of Drugs and Foods.

Clinically validated pharmacotherapeutic agents currently in clinical use were selected from DrugBank (open database of drugs, November 2017). Only drugs with FDA approval were incorporated into the model (1984 drugs out of a total of ˜10 K available in DrugBank). The DrugCentral database (open database of drugs, June 2018) was used to identify drugs designed for primary use against cancer. RepoDB (open database of repositioned drugs, November 2017) was used to identify drugs that have been successfully repositioned for anti-cancer purposes (secondary or tertiary use). For our machine-learning approach drugs designed and tested specifically for anticancer treatment (n=199) were denoted as the ‘positive’ class and drugs with no known association with cancer were used as the ‘negative’ class (n=1692). Drugs that have been repositioned for secondary/tertiary use in cancer have been excluded from the model. Drug compounds extracted from different databases were matched using InChI keys.

Drug-gene encoded protein interaction data were extracted from the STITCH database (open database of chemical-gene interactions, November 2017) and once more drug compounds were matched using InChI keys. A significance score for individual drug-protein interactions was extracted from the STITCH database. Different levels of interaction significance as defined by threshold were considered as part of the computational strategy. Compounds from FooDB (open database of foods and food compounds, June 2018) for which InChI identifier was available were matched to STITCH in the same way as drugs to generate the scored list of compound-gene interactions. The interactions were filtered according to the score threshold identical to the one used for the drugs in the model (the actual value is model-dependent). T3DB was used to highlight toxic and potentially toxic food compounds (matching performed using InChI keys).

### Compilation of Human Proteome Network Datasets

A human genome network of 20,256 proteins was compiled using data extracted from STRING, UniProt, COSMIC, and NCBI Gene public databases. Due to the heterogeneity in gene/protein nomenclature in these databases, we used a sequence-based matching approach based on protein amino acid sequence alignment to establish the correspondence between proteins across databases. The amino acid sequences of 15911 proteins out of 20,256 were precisely matched between databases. The remaining sequences were then checked to determine if any were subsets of a larger amino acid sequence in any of the above databases. This permitted further alignment of 1532 protein sequences. Finally, the remaining proteins were aligned using ‘fuzzy’ matching (allowing up to 5% amino acid sequence mismatch) generating an additional 1686 proteins. Non-matched amino acid sequences (1,127) with their corresponding database identifiers were incorporated into the unified database. This resulted in 20,256 unique gene-encoded proteins and their identifiers/names/synonyms from different databases (including Ensembl ID, HGNC), where available.

Protein-protein interactions were imported from STRING resulting in ˜11 million connections with the confidence scores in the range 0-999. Additionally, BioPlex, an open database of experimentally established protein-protein interactions, was mapped onto our gene list using gene id, Uniprot ID and gene name. ˜100 K connections for 10859 genes were added to the interactome network from BioPlex in addition to the ones imported from STRING.

Our observation showed full matching between Ensembl IDs from STRING and STITCH databases, providing a reliable link between chemical-protein and protein-protein interaction networks. Thus it was decided to use these two databases as a core model and reference for matching for other databases. Scored protein-protein interactions were imported from STRING into the propagation model with the score threshold used to filter out “unreliable” ones (adjustable parameter in the model).

### Unsupervised Learning on Graphs Using Random Walks.

The resulting interactome network was represented as a graph where nodes are gene-encoded proteins and the links between them correspond to biological interactivity. The graph makes no assumption regarding the direction of interaction between proteins (referred to as “undirected” graph). The link weights were dichotomized with various thresholds. The optimum threshold value was derived using a “nested” cross-validation strategy.

All proteins interacting with a given drug/bioactive molecule were assigned a value of 1.0 and all others were assigned the value of 0.0. This resulted in a sparse protein profile interacting with a given molecule (on average 20-30 targets per molecule). However on the understanding that these proteins act as part of the wider protein-protein network rather than in isolation, the unsupervised learning on graph algorithm (namely, a random walk with restarts) was applied to “learn” latent network-wide effects of a specific molecule. This network diffusion transforms a short list of proteins targeted by a given molecule/drug into a genome-wide profile of gene scores based on their network proximity to target candidates.

From a computational perspective, we represent targeted proteins as “entry points” for a random walk which is defined as a path consisting of a succession of random steps within the interactome network. Before the iteration starts the probability of the walker to be in any of the ‘entry’ points is set to 1.0 divided by the number of ‘entry’ points, forming the starting sparse probability distribution vector, pₒ. The probability of transition from node a to a connected node b is given by 1.0 divided by the number of outgoing connections from node a. These transition probabilities for the whole interactome form a scaled adjacency matrix, W. The probability of the walker to restart from its ‘entry’ point is given by the parameter “c”. This parameter denotes how far the influence of a given molecule spreads within the network with c=1.0 meaning no propagation beyond ‘entry’ points, while c close to 0.0 would result in potential propagation to the furthest connected node(s), resulting in a “smoother” genome-wide profile. For each subsequent step of the algorithm the new distribution of the probabilities of finding the walker in any of the nodes pᵢ is given by Eq. 1:

pᵢ=pᵢ₋₁*W*(1.0−c)+c*p₀, (1)

where pᵢ₋₁ is the probability distribution from the previous iteration. The algorithm assumes convergence when |pᵢ−pᵢ₋₁| is less than a set tolerance value and the obtained probability distribution pi (also referred to as “smoothed” genome-wide profile for a given molecule/drug) is returned for use in downstream supervised machine learning steps of the strategy.

### Supervised Machine-Learning Using Propagated Network Profiles.

Supervised-machine learning strategies based on Support Vector Machine (SVM) and Maximum Margin Criterion (“MMC”) were optimized to identify anti-cancer therapeutics based on their influence on diffused interactome profiles. The parameters for linear (“c”) and radial kernels (“c”, gamma) were optimized during SVM training. Both ‘positive’ and ‘negative’ classes of drugs formed the set used for model training. The best performing strategy (including type of interactome, parameter thresholds and settings for random walks on graphs, and supervised modeling methodology) was defined according to the F-score (balancing sensitivity and specificity) by a nested cross-validation strategy (see below). Due to the high class imbalance (˜1:9 anti-cancer vs non-anticancer drugs), F-score was used as the main measuring criterion for the performance of the classifier. Stratified K-fold and “balanced” weights were used to compensate for class imbalance. The full list of parameter combinations tried with corresponding statistics is provided in SI Dataset S1. We also trained 2 convolutional neural network classifiers and 2 regularized LASSO/Elastic Net classifiers to see whether there is any improvement in classification performance for the best performing type of interactome and settings for random walk on graphs (see Supplementary Information Appendix M1 below for methodological details).

### Overall Workflow for Drug and Active Food Molecules Repurposing.

Here, we assume that drugs/molecules acting on common protein networks (responsible for a variety of metabolic and signaling processes) should therefore exert similar downstream disease modifying effects. In order to validate this assumption and to predict unique anti-cancer compounds which could potentially be used/repositioned for cancer treatment we have tailored a bespoke machine learning strategy as outlined below:
(1) The proteins interacting with molecular compounds (either existing drugs or bioactive compounds within foods) were mapped onto interactome;
(2) The network-wide diffused effect of a given molecule was derived using a grid of different settings: the type of interactome network (BioPlex or STITCH), varying connection thresholds for the links between proteins (STRING, STITCH and BioPlex interactomes), and varying values of the “c” parameter in the random walk propagation algorithm);
(3) A supervised-machine learning strategy based on SVM, MMC and CNN algorithms was optimized to identify anti-cancer therapeutics based on their influence on diffused interactome networks.
(4) Molecular anti-cancer “likeness” was calculated as the probability outcome of the best performing ML strategy (F-score≥0.84, achieved by the 700 best performing models). These anti-cancer probability estimates were used to create a summary table of potential candidates for anti-cancer repurposing (Supplementary Information Dataset S2 in Veselkov et al. 2019).
(5) Once validated on anti-cancer therapeutics, food compounds were processed in exactly the same way as the drugs used to train the models and then the best models obtained in the previous step were used to generate probabilistic predictions for the anti-cancer “likeness” of these food compounds (Supplementary Information Dataset S3 in Veselkov et al. 2019).
(6) The list of the food compounds with the highest probability of exhibiting anti-cancer properties has been compiled and manually curated to exclude toxic compounds and compounds shown to promote cancer (the model is effective at highlighting both anti-cancer compounds and cancer-promoting compounds as they often share underlying biological mechanisms and interactions). Furthermore, compounds associated with normal metabolism of cells, e.g. dCTP belonging to the superclass of nucleosides, nucleotides, and analogues and directly involved in DNA synthesis were also removed from the final curated list. The compound-food associations were retrieved from the FooDB database. The curated results are provided as Supplementary Information Appendix Tables 1&2 in Veselkov et al. 2019 and FIGS. 12 and 13.

### Nested Cross-Validation Strategy.

A 10-fold nested cross-validation strategy was employed to assess the predictive capacity of each method and model generated. Each test and training set split was stratified to keep equal proportions of ‘positive’ (anti-cancer therapeutics) and ‘negative’ (non anti-cancer therapeutics) classes in each split. For linear and radial SVM classifiers 5-fold inner cross-validation was used to optimize C and gamma parameters. Average per class classification accuracy and F-score metrics were used for the assessment of model predictive capacity due to class imbalance (˜1:9 for ‘positive’:‘negative’ classes). Logistic regression was employed for MMC as well as linear and radial SVMs to provide classification probability estimates. For each fold the anti-cancer “likeness” of a given molecule (based on its influence on interactome networks) in the test set was predicted. Averaged F-scores from 10-fold outer cross-validation was used to select the best ML strategy among all combinations of pre-processing, unsupervised and supervised model parameters (drug-gene connection confidence thresholds: 0, 100, 200, 325, 400, 500, 600, 700; gene-gene connection confidence thresholds: 400, 600, 700, 800, 850 or present in BioPlex; Random walk with restarts “c”: 0.0001, 0.001, 0.002, 0.004, 0.01, 0.015, 0.02, 0.03, 0.035, 0.04, 0.05, 0.076, 0.1, 0.2; preprocessing with log-transform: yes/no). The models were re-trained using the entire set of ‘positive’ and ‘negative’ classes (and the averaged best C and gamma, where applicable) prior to using them to predict anti-cancer “likeness” of the food compounds and the drugs which were not a part of the model building set. All tested parameterization sets and training statistics are provided in the Supplementary Information Dataset S1 in Veselkov et al. 2019.

### Pathway Analytics.

Pathway analytics was performed using gene set enrichment analysis via Python GSEAPY package 61. Propagated gene/protein perturbation values were supplied as the input data for “prerank” module. Reactome_2016 and KEGG_2016 gene sets were used by default. Scored pathways were sorted by the normalized enrichment score reported by the script. Top 10 pathways for each gene collection and each CBM were reported in SI Dataset S3 in Veselkov et al. 2019.

### Supplementary Methods (M1): Justification for the Use of Linear SVM and MMC

We also trained 2 neural networks and regularized LASSO/Elastic Net classifiers to see whether there is any improvement in classification performance for the best performing type of interactome and settings for random walk on graphs. The first NN-1 classifier had a fully-connected layer with a 2-dimensional output and softmax activation function to output probabilities of belonging to anticancer and non-anticancer classes. The second NN-2 classifier comprised a linear layer (with an output dimensionality of number of molecules-1) and a fully-connected layer (with a 2-dimensional output) with softmax activation function. Both classifiers were trained using Momentum optimizer and l2 regularization. We used weighted cross-entropy as the cost function. Model performance was evaluated using 10-fold cross-validation. In the cross-validations, the training data was further split into training and validation set (10%), using the validation set for early stopping: training was stopped when either (i) the maximum number of epochs was reached (20K) or (ii) the validation loss continuously increased in a window of 5 evaluation steps (with evaluations every 50 epochs). For each fold, the model was saved when the validation loss was lowest and used for prediction on the test set. Cross-2 validation experiments were done to find the optimal learning rate and l2 regularization hyper-parameter. Optimal values of learning rate and l2 regularization parameters were 10 and 1e-4 for the first classifier, and 1e-2 and 1 for the second classifier. Finally, regularized LASSO and Elastic Net classifiers were trained using stochastic gradient decent. The model parameters (alpha for LASSO and alpha/l1 for Elastic Net) were optimized using 10 fold nested cross validation. Final results (F-score) in 1:1 comparison were as follows:
1) LinearSVM: 84.7%
2) RadialSVM: 84.0%
3) LASSO: 82.7%
4) NN model 2: 81.3%
5) NN model 1: 80.1%
6) LASSO_logreg: 77.5%
7) Elastic Net: 72.9%
8) Elastic Net_logreg: 70.0%

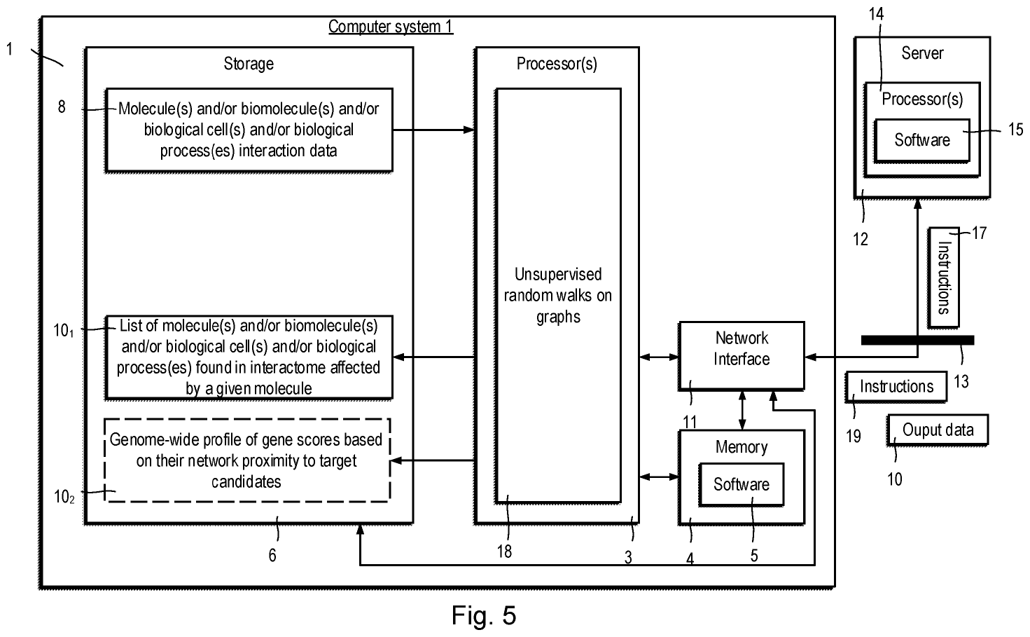

Referring to FIG. 5, a first computer system 1 includes at least one processor 3 and memory 4 operatively connected to the processor 3. The memory 4 may include software 5. The software 5 may include instructions to perform one or more methods described herein.

The system 1 includes storage 6. The storage 6 may store input data 8 and output data 10. Input data 8 may be, for example, molecule(s) and/or biomolecule(s) and or biological cell(s) and/or biological process(es) interaction data.

Interaction data may include interaction data between a molecule(s) and a molecule(s), interaction data between a molecule(s) and a biomolecule(s), interaction data between a molecule(s) and a biological cell(s), or a molecule(s) and interaction data between a biological process(es). Interaction data may include interaction data between a biomolecule(s) and a biomolecule(s), interaction data between a biomolecule(s) and a biological cell(s) or interaction data between a biomolecule(s) and a biological process(es). Interaction data may include interaction data between a biological cell(s) and a biological cell(s), interaction data between a biological cell(s) and a biological process(es). Interaction data may include interaction data between a biological process(es) and a biological process(es). Interaction data may further include interaction data between a biological entity/entities and a biological entity/entities, interaction data between a biological entity/entities and a molecule, interaction data between a biological entity/entities and a biomolecule(s), interaction data between a biological entity/entities and a biological cell(s) and interaction data between a biological entity/entities and a biological process(es). Interaction data may also include interactions between one or more element(s), for example, hydrogen, iron, zinc or lithium, and any one or combination of a molecule(s), biomolecule(s), a biological cell(s) or a biological process(es).

Interaction data may be in vivo interaction data. Interaction data may be in vitro interaction data. Interaction data may be interaction data related to a biological process(es).

A first output data 101 may include, for example, a list of molecule(s) and/or biomolecule(s), and/or biological cell(s) and/or biological processes found in an interactome network that are affected by a given (input) molecule(s). A second output data 102 may include data relating to a genome-wide profile of gene scores based on their network proximity to target candidates.

The first computer system 1 may have a network interface 11 connected to a server 12 via a network 13 or network connection. The network interface 11 may be connected to at least the processor(s) 3, the storage 6 and the memory 4. The network connection may be a local network or a global network. The network connection may be a Local Area Network (LAN), or the internet. The network connection may be a wireless connection, for example a Wireless Wide Area Network (WAN) or a cellular network. The server 12 may include one or more processors 14 which run application software 15, the server application software may be, for example DreamLab App. The server 12 may pass instructions 17 from the server software 14 to the memory 4. These instructions 17 are then passed to the processor 3. The instructions 17 may be instructions to get more instructions from the software 5 on the memory 4. The instructions may be to run a model 18 on the processor 3 which uses the input data 8 and outputs the output data 10. The model 18 may be, for example, unsupervised random walks on graphs. The first computer system 1 may pass instructions 19 and output data 10 to the server 12 via the network. Based on these instructions 19 and output data 10, the software application 15 on the server may send more instructions 17 to the first computer system.

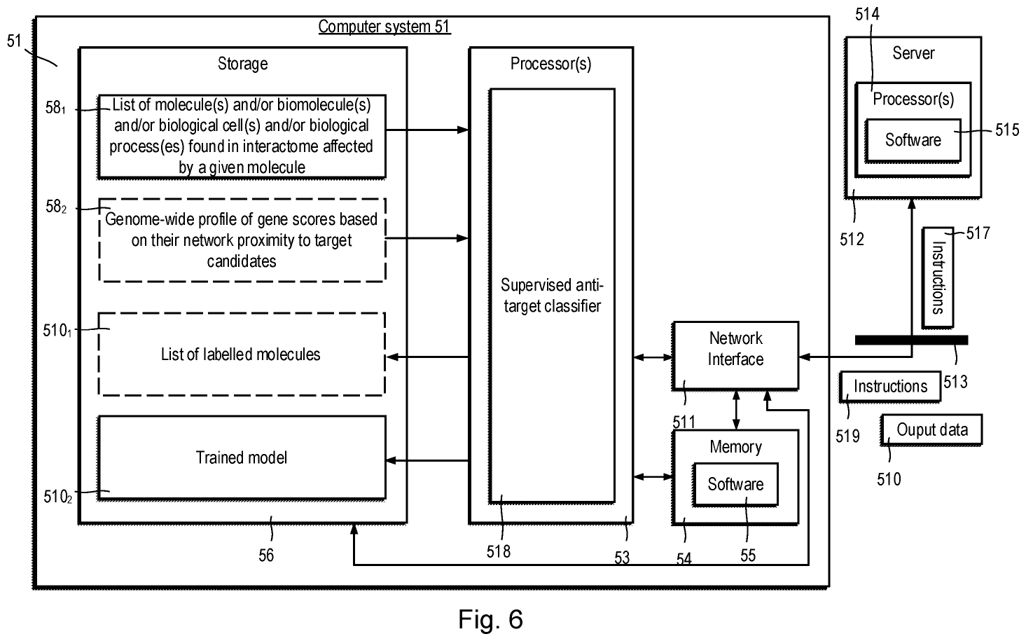

Referring to FIG. 6, a second computer system 51 includes at least one processor 53 and memory 54 operatively connected to the processor 53. The memory 54 may include software 55. The software 55 may include instructions to perform one or more methods described herein.

The second computer system 51 includes storage 56. The storage 56 may store input data 58 and output data 510. A first input data 58₁ may be, for example, a list of molecule(s) and/or biomolecule(s) and/or biological cell(s) and/or biological process(es) found in interactome affected by a given molecule. Input data 58₂ may further include genome-wide profile of gene scores based on their network proximity to target candidates.

In the second computer system 51, a first output data 5101 may include, for example, a list of (labelled) molecule(s). A second output data 510₂ may be a trained model.

The second computer system 51 may have a network interface 511 connected to a server 512 via a network 513 or network connection. The network interface 511 may be connected to at least the processor(s)₅₃, the storage 56 and the memory 54. The network connection may be a local network or a global network. The network connection may be a Local Area Network (LAN), or the internet. The network connection may be a wireless connection, for example a Wireless Wide Area Network (WAN) or a cellular network. The server 512 may include application software 514, the server application software may be, for example DreamLab App. The server 512 may include one or more processors 514 which run application software 515, the server application software may be, for example DreamLab App. The server 512 may pass instructions 517 from the server software 514 to the memory 54. These instructions 517 are then passed to the processor 53. The instructions 517 may be instructions to get more instructions from the software 55 on the memory 54. The instructions may be to run a model 518 on the processor 53 which uses the input data 58 and outputs the output data 510. The model 518 may be, for example, unsupervised random walks on graphs. The first computer system 51 may pass instructions 519 and output data 510 to the server 512 via the network. Based on these instructions 19 and output data 510, the software application 515 on the server may send more instructions 517 to the first computer system.

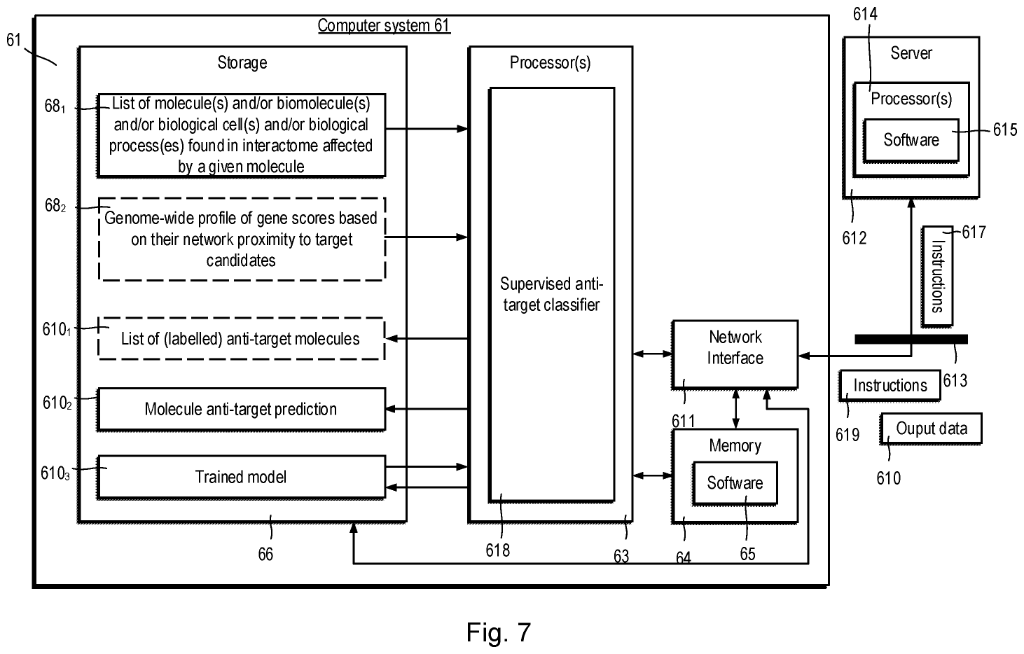

Referring to FIG. 7, a third computer system 61 includes at least one processor 63 and memory 64 operatively connected to the processor 63. The memory 64 may include software 65. The software 65 may include instructions to perform one or more methods described herein.

The second computer system 61 includes storage 66. The storage 66 may store input data 68 and output data 610. A first input data 68₁ may be, for example, a list of molecule(s) and/or biomolecule(s) and/or biological cell(s) and/or biological process(es) found in interactome affected by a given molecule. Input data 68₂ may further include genome-wide profile of gene scores based on their network proximity to target candidates.

In the second computer system 61, a first output data 610₁ may include, for example, a list of (labelled) molecule(s). A second output data 610₂ may be, for example, a molecule(s) anti-target prediction. The prediction may be probabilistic. A third output data 610₃ may be a trained model. In the third computer system, the trained model output 610₃ may also be used as an input to classify further molecules.

The third computer system 61 may have a network interface 611 connected to a server 612 via a network 613 or network connection. The network interface 611 may be connected to at least the processor(s) 63, the storage 66 and the memory 64. The network connection may be a local network or a global network. The network connection may be a Local Area Network (LAN), or the internet. The network connection may be a wireless connection, for example a Wireless Wide Area Network (WAN) or a cellular network. The server 612 may include application software 614, the server application software may be, for example DreamLab App. The server 612 may include one or more processors 614 which run application software 615, the server application software may be, for example DreamLab App. The server 612 may pass instructions 615 from the server software 614 to the memory 64. These instructions 617 are then passed to the processor 63. The instructions 617 may be instructions to get more instructions from the software 65 on the memory 64. The instructions may be to run a model 618 on the processor 63 which uses the input data 68 and outputs the output data 610. The model 618 may be, for example, unsupervised random walks on graphs. The first computer system 1 may pass instructions 619 and output data 610 to the server 612 via the network. Based on these instructions 619 and output data 610, the software application 615 on the server may send more instructions 617 to the first computer system.

The first, second and third computer systems 1, 51, 61 may be any suitable computer system. They may be, for example, a desktop PC or laptop. They may be a smartphone or tablet device. The first, second and third computer systems 1, 51, 61 may be separate devices. Alternatively, the first, second and third systems 1, 51, 61 may also be the same device, and may perform the methods outlined herein sequentially or in parallel. The first, second and third servers 12, 512, 612 may be any suitable serve, they may be cloud-based server.

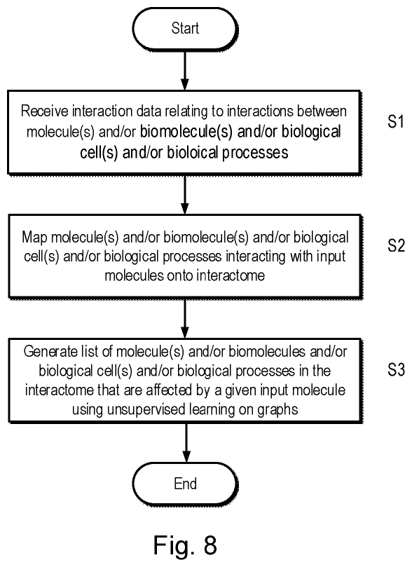

Referring to FIG. 8, a list of molecule(s) and/or biomolecules and/or biological cell(s) and/or biological processes in an interactome that are affected by a given (input) molecule is generated using unsupervised learning on graphs. Interaction data relating to interactions between molecule(s) and/or biomolecule(s) and/or biological cell(s) and/or biological processes is received (step S1). Molecule(s) and/or biomolecule(s) and/or biological cell(s) and/or biological processes interacting with input molecules are then mapped onto an interactome network. The interactome network is a graph comprising node(s) and node link(s), wherein each node is a molecule, a biomolecule, a biological cell and/or a biological process and each node link corresponds to interactivity (step S2). For a given input molecule, a list of molecule(s) and/or biomolecules and/or biological cell(s) and/or biological processes in the interactome that are affected by the given input molecule is generated using unsupervised learning on graphs (step S3).

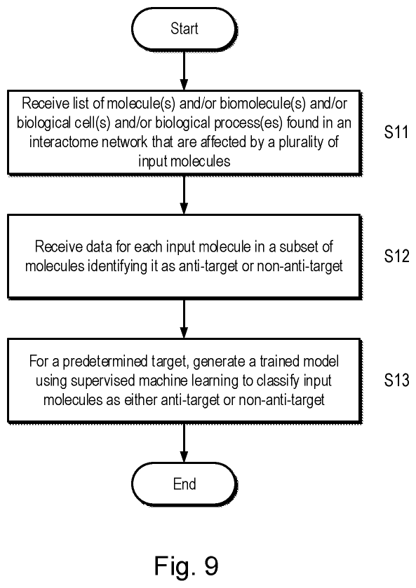

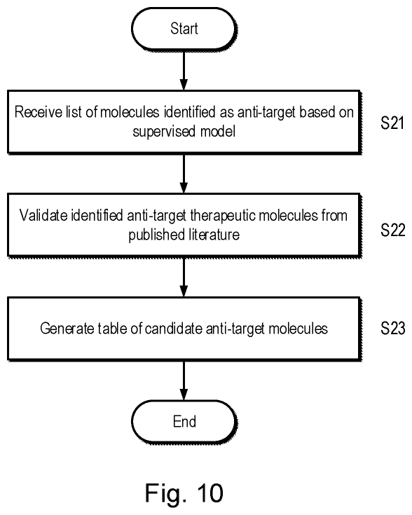

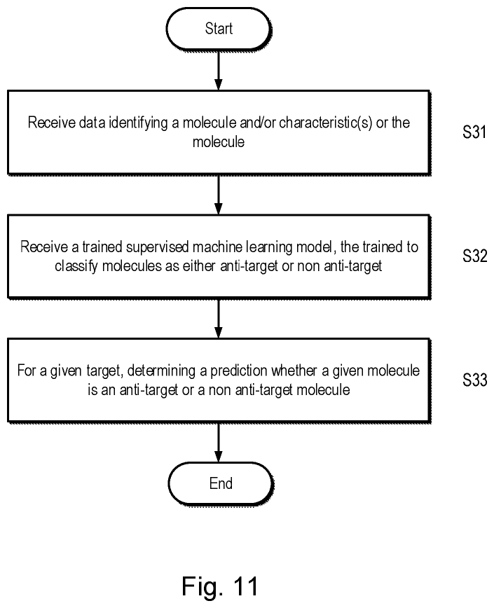

Referring to FIG. 9, for a pre-determined target, a trained model is generated using supervised machine learning which classifies (input) molecules as either anti-target or non-anti-target input molecules. A list of a molecule(s) and/or a biomolecule(s) and/or a biological cell(s) and/or a biological process(es) found in an interactome network that are affected by a plurality of input molecules is received (step S11). Data identifying or labelling each (input) molecule in a sub-set of the plurality of input molecules as an anti-target input molecule or a non-anti-target (input) molecule is received (step S12). A trained model 22 is generated using supervised machine learning, and the ground-truth data for the input molecules provided by the input molecule identity or label. The model is trained to classify input molecules as either anti-target or non-anti-target based on the influence of the input molecules on diffused the interactome networks (step S13).

Referring to FIG. 10, a validated table of anti-target input molecules is generated. A list of (input) molecule(s) identified as anti-target (input) molecule(s) classified using the train model 22 is received (step S21). The identified anti-target input molecules are validated as therapeutic molecules using natural language processing to assess the identified molecules in the published literature (step S22). Those input molecules which are confirmed as anti-target molecules from the published literature are then output in a list or table (step S23).

Referring to Figure ii, for a given target, a prediction whether an input molecule(s) is an anti-target or a non-anti-target input molecule(s) is generated using a trained model. Data identifying an input molecule(s) and/or characteristic(s) of the input molecule(s) is received (step S31). A trained supervised machine learning model, the trained model generated using a supervised machine learning strategy to classify (input) molecules as either anti-target or non-anti-target based on the influence of the molecules on diffused an interactome networks of a molecule(s) and/or a biomolecule(s) and/or a biological cell(s) and/or a biological process(es) is received (step S32). Using the trained model, for a given target, a prediction whether the input molecule(s) is an anti-target or a non-anti-target candidate input molecule(s) is determined (step S33).

Modifications

It will be appreciated that various modifications may be made to the embodiments hereinbefore described. Such modifications may involve equivalent and other features which are already known in the design and use of determining molecule effect methods, systems and component parts thereof and which may be used instead of or in addition to features already described herein. Features of one embodiment may be replaced or supplemented by features of another embodiment.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A computer-implemented method comprising:
receiving interaction data relating to interactions between a molecule(s) and/or a biomolecule(s) and/or a biological cell(s) and/or a biological process(es);
generating an interactome network by mapping the molecule(s) and/or biomolecule(s) and/or biological cell(s) and/or biological process(es) interacting with an input molecule(s) onto a graph comprising node(s) and node link(s), wherein each node is a molecule(s) and/or a biomolecule(s) and/or a biological cell(s) and/or a biological process(es) and each node link corresponds to interactivity; and
generating a list of a molecule(s) and/or a biomolecule(s) and/or a biological cell(s) and/or a biological process(es) found in the interactome network that are affected by an input molecule by using unsupervised learning on graphs to identify latent network-wide effects of the given input molecule.

2. The method of claim 1 wherein the type of interactome network is experimentally derived and/or computationally predicted.

3. The method of claim 1 wherein the unsupervised learning on graphs is a random walk with a diffusion kernel or operator.

4. The method of claim 1 wherein the unsupervised learning on graphs further comprises varying parameters of the interactome and varying parameters of diffusion algorithms.

5. The method of claim 1 further comprising generating a genome-wide profile of gene scores based on gene interactome network proximity to molecule target candidates.

6. The method claim 3 wherein the entry node for a random walk represents a targeted molecule(s) and/or a targeted biomolecule(s) and/or a targeted biological cell(s) and/or a targeted biological process(es).

7. The method of claim 1 further comprising simulating the perturbation of one or more input molecule(s) through the interactome network using the input molecule(s) interaction data; and
outputting the interactions the of the input molecule in the network.

8. The method of claim 1 wherein the input molecule(s) is a molecule(s) in an existing drug(s) or a bioactive compound(s) in food.

9. The method of claim 1 further comprising generating a sparse molecules(s) and/or biomolecule(s) and/or biological cell(s) and/or biological process(es) profile interacting with an input molecule by assigning a value of 1 to all molecules(s) and/or biomolecule(s) and/or biological cell(s) and/or biological process(es) in the interactome that interact with the input molecule and assigning a value of 0 to all other molecules(s) and/or biomolecule(s) and/or biological cell(s) and/or biological process(es).

10. A computer implemented method comprising:
receiving a list of a molecule(s) and/or a biomolecule(s) and/or a biological cell(s) and/or a biological process(es) found in an interactome network that are affected by a plurality of input molecules, each input molecule in a sub-set of the plurality of input molecules being identified as an anti-target input molecule or a non-anti-target input molecule;
for a predetermined target, generating a trained model using supervised machine learning to classify input molecules as either anti-target or non-anti-target based on the influence of the input molecules on the interactome network.

11. The method of claim 10 wherein the influence of the input molecule(s) on an interactome network may be determined by applying at least one layer of parametric diffusion to the input molecule(s) data on the molecule(s) and/or biomolecule(s) and/or biological cell(s) and/or a biological process(es) interactome.

12. The method of claim 11 wherein the parameters of parametric diffusion are determined by training.

13. The method of claim 12 wherein the training procedure comprises:
receiving a training dataset of input molecules, the dataset comprising a molecule interaction signal and the molecule ground-truth property for each molecule; and
tuning the parameters to optimize a loss function.

14. The method of claim 13 wherein the training dataset of input molecules further includes a molecule chemical descriptor for each input molecule(s).

15. The method of claim 13 wherein the loss function comprises at least one selected from the group consisting of:
a distance between the predicted input molecule properties and the ground-truth input molecule properties; or
a classification error.

16. A computer implemented method comprising:
receiving data identifying an input molecule(s) and/or characteristic(s) of the input molecule(s);
receiving a trained supervised machine learning model, the trained model generated using a supervised machine learning strategy to classify an input molecule(s) as either anti-target or non-anti-target based on the influence of the input molecule(s) on an interactome network of a molecule(s) and/or a biomolecule(s) and/or a biological cell(s) and/or a biological process(es);
for a given target, determining, using the trained model, a prediction whether the input molecule(s) is an anti-target or a non-anti-target input molecule(s).

17. The method of claim 16 wherein the data relating to the input molecule is interactome network-wide diffused effect data.

18. The method of claim 16 wherein the data relating to the input molecule includes a simulated perturbation of the molecule through interactome network-wide diffused effect data.

19. The method of claim 1 further comprising calculating the anti-target probability outcome of the best performing learning strategy for the given input molecule.

20. The method of claim 1 further comprising:
for an input molecule determined as anti-target:
extracting information relating to the input molecule and information relating to the input molecule therapeutic effects from a database using natural language processing; for the given target, determining whether the input molecule is a confirmed anti-target molecule.

21. The method of claim 16 further comprising outputting a list of confirmed anti-target molecules.

22. A computer system comprising:
at least one processor; and
memory;
wherein the memory stores computer readable instructions that, when executed by the at least one processor, causes the computer system to perform the method of claim 1.

23. The system of claim 22 further comprising storage for storing interaction data and/or an interactome and/or a list of molecule(s) and/or biomolecule(s) and/or a biological cell(s) and/or a biological process(es) and/or a trained model.

24. A non-transitory computer readable medium which stores a computer program which comprises instructions for performing a method according to claim 1.
